(19) United States
(12) Patent Application Publication
(10) Pub. No.: US 2020/0308184 A1
(43) Pub. Date: Oct. 1, 2020

(54) TETRAPYRROLIC CONJUGATES AND USES THEREOF FOR IMAGING

(71) Applicant: Health Research, Inc., Buffalo, NY (US)

(72) Inventors: Ravindra K. PANDEY, East Amherst, NY (US); Zachary GROSSMAN, Buffalo, NY (US); Ravindra CHERUKU, Buffalo, NY (US); Joseph MISSERT, Buffalo, NY (US); Joseph SPERNYAK, West Seneca, NY (US); Craig M. HENDLER, Buffalo, NY (US); Ronald A. ALBERICO, Buffalo, NY (US); Sandra SEXTON, Buffalo, NY (US); Farukh DURRANI, East Amherst, NY (US)

(21) Appl. No.: 16/649,582

(22) PCT Filed: Sep. 21, 2018
(86) PCT No.: PCT/US2018/052282

§ 371 Date: Mar. 20, 2020

Related U.S. Application Data
(60) Provisional application No. 62/561,503, filed on Sep. 21, 2017.

(51) Int. Cl. C07D 487/22; A61K 41/00; A61K 49/00; A61K 49/08

(52) U.S. Cl. CPC C07D 487/22; A61K 41/0071; A61K 49/085; A61K 49/0036; A61K 49/0052; A61K 49/0002

(57) ABSTRACT

The present disclosure provides compounds comprising a tetrapyrrole (e.g., a tetrapyrrole group) linked via a linker to one or more ligands and uses of the compounds. The compounds may be used as imaging agents (e.g., MRI contrast agents) or as both imaging and therapeutic agents. The compounds may be used to treat individuals in need of treatment for a hyperproliferative disorder, such as, for example, malignancy. The present disclosure also provides kits comprising pharmaceutical preparations containing any one or any combination of the compounds described herein.

### CROSS REFERENCE TO RELATED APPLICATIONS

This application claims priority to U.S. Provisional Application No. 62/561,503, filed on Sep. 21, 2017, the disclosure of which is hereby incorporated by reference.

### FIELD OF THE DISCLOSURE

The disclosure generally relates to tetrapyrrolic conjugates and uses thereof. More particularly the disclosure relates to uses of tetrapyrrolic conjugates in therapeutic and imaging methods.

### BACKGROUND OF THE DISCLOSURE

Magnetic resonance imaging (MRI) is a versatile medical imaging technique that provides multiple applications in medicine, including oncological (cancer) imaging. MRI provides superb spatial resolution and yields unparalleled soft tissue contrast in living subjects that can be further augmented with the use of contrast-enhancing agents. This makes MRI highly useful for obtaining detailed morphological information for solid tumors. Unlike PET or CT, MRI does not use ionizing radiation, which in turn reduces potential health risks to patients during imaging sessions. Furthermore, the depth of penetration into the living subjects is a non-issue for MRI, a significant advantage over optical fluorescence and ultrasound imaging. However, MRI has only moderate sensitivity in certain tissues, scanning is slow, and it is more expensive than CT. MRI sensitivity is greatly increased by intravenous injection of Gd-based contrast media, but the currently available contrast agents clear rapidly from the circulation by glomerular filtration, and as they are excreted, tumor conspicuity fades in 20-30 minutes.

In recent years, designing bi-functional agents based on certain porphyrin-based compounds has been a focus; the two functions, provided by each agent, are (1) tumor imaging and (2) photodynamic therapy (PDT). PDT is localized therapy that works when a tumor avid agent is administered, and after uptake of the agent by the tumor, the tumor is exposed to a specific wavelength of laser light, which engenders the creation of cytotoxic lethal singlet oxygen in the tumor. One of the first generation photosensitizers approved by the FDA was porfimir sodium (Photofrin), which has been shown to be effective against Barrett's esophagus, cervical, endobronchial, and papillary bladder cancers. However, Photofrin has two main limitations: Photofrin cannot absorb energy efficiently at longer wavelengths (635 nm), which limits its effectiveness against deep-seated tumors; and aside from localizing within the tumor, Photofrin localizes in skin uptake, resulting in unwanted skin photosensitivity. These limitations led to the development of second generation photosensitizers that are more tumor-avid and are activated by light of longer wavelength. One such photosensitizer, developed in our laboratory is 3-(1-hexyloxyethyl) pyropheophorbide-a (HPPH), derived from chlorophyll-a. It exhibits long wavelength peak absorption at 665 nm in vivo, and is currently undergoing Phase II clinical trials of head & neck cancer (SCC). Furthermore, unlike many porphyrin-based compounds, HPPH has a relatively high fluorescence quantum yield which makes it a suitable fluorophore for optical fluorescence imaging. Therefore, HPPH presents itself as a promising bi-functional agent fluorescence-guided surgery and PDT of certain types of cancer.

It has been previously shown that certain tumor-avid compounds, including HPPH, can function as a delivery vehicle for cancer therapeutic agents to tumor, and also as MR imaging agents; hence, their “multi-functional” status. This multi-functionality has created a potential for “see and treat” in one compound, and represents new paradigm for dealing with certain cancers.

Earlier work converted HPPH into a series of mono- di- and tri-Gd(DTPA) conjugates; the 3GdDTPA moiety was the most the effective MR imaging agent (more gadolinium atoms on the molecule created greater signal). However, its solubility in most of the US FDA accepted formulations was extremely low. Replacing three DTPA with three DOTA moieties significantly enhanced water solubility, retained its tumor avidity, fluorescence imaging potential (in vivo), PDT efficacy (in vitro), and its tumor enhancement on MRI.

Although the general public generally considers medical and surgical therapy the bedrock of cancer treatment, radiologists and pathologists have argued for 50 years that if they do not know what a lesion is, and where it is, then they as therapists are lost. Defining the malignant process, and its extent, is the heart of cancer staging.

Thus, beginning with plain film radiography, then fluoroscopy, contrast angiography, CT, nuclear imaging, MRI, and now both PET/CT and MRI/PET, technology for localizing tumors in patients has advanced. For over 15 years, F-18-FDG-PET/CT has been the “gold standard” for defining widespread metastases and, therefore, for cancer staging, but very recently F-18-FDG-PET/MRI has been gaining ground. However, there is a long felt and unmet need for better imaging and photodynamic therapy compounds.

### SUMMARY OF THE DISCLOSURE

The present disclosure provides compounds and uses of the compounds. For example, the compounds are used as MRI contrasts agents.

In an aspect, the present disclosure provides compounds. The compounds of the present disclosure can be made by methods disclosed herein. The compounds can be used as is described herein.

A compound comprises a tetrapyrrole (e.g., a tetrapyrrole group) linked via a linker to one or more ligands. In various examples, a compound comprises: F-L-R, where F is a tetrapyrrole (e.g., a porphyrin, such as, but not limited to, HPPH and derivatives and analogs thereof), optionally, comprising a chelating ion (e.g., deuterium (²H), ⁶⁴Cu, ¹¹¹In, ⁵⁷Ga), L is a linker, and R is a ligand, optionally, comprising a Gd(III) ion coordinated to the ligand.

In various examples, the compound is a salt, a partial salt, a hydrate, a polymorph, an isomer (e.g., a structural or stereoisomer), or a mixture thereof. The compounds can have stereoisomers. For example, the compound can be present as a racemic mixture, a single enantiomer, a single diastereomer, mixture of enantiomers, or mixture of diastereomers.

The compounds of the present disclosure include pharmaceutically acceptable derivatives and prodrugs of the compounds of the present disclosure. A compound may be a lyophilized compound (e.g., a lyophilized powder).

In an aspect, the present disclosure provides compositions comprising one or more compound of the present disclosure. The compositions may comprise one or more pharmaceutically acceptable carrier.

In an aspect, the present disclosure provides uses of compounds of the present disclosure. The compounds can be used as imaging agents (e.g., MRI contrast agents) or as both imaging and therapeutic agents.

In various examples, the present disclosure provides methods that use one or more compounds of the present disclosure. Examples of methods include, but are not limited to, methods of imaging an individual (or a portion thereof) and methods of imaging and treating an individual.

This disclosure provides methods of treating individuals in need of treatment (e.g., for a hyperproliferative disorder, such as, for example, malignancy (e.g., a malignancy disorder)) comprising administering to an individual a compound or composition of the present disclosure, and imaging the individual or a portion thereof and, after staging the disease, proceeding to appropriate therapy (e.g., surgical, chemotherapeutic, photodynamic, or standard radiation).

In another aspect the disclosure further provides products, e.g. articles of manufacture such as for example, kits, which comprise pharmaceutical preparations containing any one or any combination of the compounds described herein.

### BRIEF DESCRIPTION OF THE FIGURES

For a fuller understanding of the nature and objects of the disclosure, reference should be made to the following detailed description taken in conjunction with the accompanying figures.

FIG. 1 shows a standard curve of Gd determined using Xylenol Orange assay. Free Gd concentration of HPPH-Dotaₙ-Gdₙ samples.

FIG. 2 shows an overlay of Xylenol Orange test spectra of Gadolinium Standards -Free Gd³⁺=0, 10, 20, 30, 40 and 50 μM.

FIG. 3 shows an overlay of Xylenol Orange Test of filtrate of spin-filtration of HPPH-Dota1Gd1 solution with 0 and 10 μM Gd³⁺/Xylenol Orange spectra.

FIG. 4 shows an overlay of Xylenol Orange Test of filtrate of spin-filtration of HPPH-Dota2Gd2 solution with 0 and 10 μM Gd³⁺/Xylenol Orange Spectra.

FIG. 5 shows an overlay of Xylenol Orange Test of filtrate from spin-filtration of HPPH-Dota3Gd3 solution with 0 and 10 μM Gd⁺/Xylenol Orange spectra.

FIG. 6 shows an overlay of Xylenol Orange Test of filtrate from spin-filtration of HPPH-Dota3Gd3 Solution with 0 and 10 μM Gd³⁺/Xylenol Orange spectra.

FIG. 7 shows theoretical enhancement of tumor tissue (T1/T2 times=100/2000 ms) signal at 4.7T with T1W-SPGR scan (TE/TR, FA=3/15 ms, 60°).

FIG. 8 shows normalized signal intensities for tumor and organs using T1-weighted, spoiled-gradient echo scans (a-c). Maximum tumor signal correlates maximum tumor T1 rate at 4 hours post-injection (d).

FIG. 9 shows representative slices from a fat-suppressed, 3D-SPGR scan before, 15 minute, 4 hours and 24 hours post-administration of agent. Selected tumor growth (pseudo-colorized) showed maximum enhancement at 4 hours with continued enhancement up to 24 hours after administration. The strongly enhanced urinary bladder (B) has been labelled to prevent image misinterpretation.

FIG. 10 shows a comparison of photographic (left), fluorescence overlay (middle) and three-dimensional volumetric rendering of MR data (right). Mesenteric growth visible on the photographic image (arrowheads) showed strong residual fluorescence at 24 hrs. Fluorescence imaging demonstrated good spatial correlation to growths identified in MRI scans. Liver is denoted as dark red for spatial reference.

FIG. 11 shows necropsy of rabbit, 3 weeks after intra-arterial injection of VX2 tumor cells. Note innumerable tumor implants in multiple organs.

FIG. 12 shows 30 min after peripheral intravenous F-18-FDG injection, F-18-FDG-PET revealed multiple metastatic tumors (FIG. 12A, F-18-FDG-PET, and FIG. 2B, Fused F-18-FDG-PET/CT). Note hyper-metabolism in multiple, scattered, discrete sites, all corresponding to metastatic tumor on necropsy. Conspicuity of lesions on the PET and even fused PET/CT images is excellent, but resolution of anatomic detail is poor.

FIG. 13 shows left: the large size of the central auricular artery of an adult male rabbit, compared, side-by-side, to a 20 gauge hypodermic needle. Right: arrows demonstrate the rabbit aorta, after retrograde injection of radiographic contrast medium into the very large auricular artery; it is this route that we used to achieve systemic dissemination of cancer cells (original tumor dissemination model); the aorta connects directly to the cardiac left ventricle, and cells are then pumped out into the systemic circulation and disseminated widely (multiple organs, FIG. 11, FIG. 12).

FIG. 14 shows (A): Baseline before injection of 3Gd(DOTA)HPPH. (B): Immediately post injection, barely-visible lesions. (C): 6 h after injection: two soft tissue lesions are becoming visible (D): at 24 h the lesions are more conspicuous that at 6 h, as the lesions progressively accumulate pharmaceutical.

FIG. 15 shows (A): Baseline: before injection of 3Gd(DOTA)HPPH. (B): Immediately post injection: skull base lesion is visible, because of high vascularity. (C and D): 6 and 24 h post injection: Persistent enhancement of the metastasis.

FIG. 16 shows virtually concurrent trans-axial images, through the rabbit pelvis and part of the leg, reveal the strengths and weaknesses of three high-technology systems. Upper left: F-18-FDG-PET/CT. Great lesion conspicuity, but no spatial resolution. Upper middle: CT. Excellent spatial resolution, but no conspicuity; metastasis is invisible. Upper right: MRI. Metastatic lesion is visible, with good detail—i.e., conspicuity and resolution.

FIG. 17 shows tumor specificity of HPPH-3Gd(DOTA) in rats. The ward tumor cells were transplanted by injecting rats intraperitoneally, and the fluorescence imaging was performed at day 7 post-injecting the cells.

FIG. 18 shows in vivo tumor, liver and skin uptake of HPPH 1 (Gd-DOTA) conjugate (A) and HPPH-2Gd DOTA conjugate (B). Excitation: 640 nm, Emission >720 nm. Dose: 5 μmol/kg.

FIG. 19 shows in vivo uptake (fluorescence) of HPPH-3DOTA in Balb/c mice bearing Colon 26 tumors: Excitation: 640 nm, Emission: >720 nm. Dose: 5 μmol/kg.

FIG. 20 shows in vitro PDT activity HPPH Gd(DTPAT) conjugates via the cell viability MTT assay in Colon26 cells (for details see the text). Values are expressed as cell percent survival. Data represents the average of at least 3 experiments, error bars represent standard deviation.

FIG. 21 shows a coronal slice 6 h after IV injection of 3Gd(DOTA)-HPPH. Multiple base of skull and neck soft tissue metastases (indicated by arrows).

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range. All ranges provided herein include all values that fall within the ranges to the tenth decimal place, unless indicated otherwise.

The present disclosure provides compounds and uses of the compounds. For example, the compounds are used as MRI contrasts agents.

As used herein, unless otherwise stated, the term “group” refers to a chemical entity that has one terminus that can be covalently bonded to other chemical species. Examples of groups include, but are not limited to:

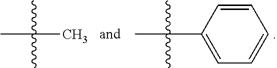

As used herein, unless otherwise indicated, the term “alkyl” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, the alkyl group can be a C₁ to C₁₂, including all integer numbers of carbons and ranges of numbers of carbons therebetween, alkyl group. The alkyl group can be unsubstituted or substituted with one or more substituent. Examples of substituents include, but are not limited to, various substituents such as, for example, halogens (—F, —Cl, —Br, and —I), aliphatic groups (e.g., alkyl groups, alkenyl groups, and alkynl groups), aryl groups, alkoxide groups, carboxylate groups, carboxylic acids, ether groups, and the like, and combinations thereof.

As used herein, unless otherwise indicated, the term “heteroalkyl” refers to branched or unbranched saturated hydrocarbon groups comprising at least one heteroatom. Examples of suitable heteroatoms include, but are not limited to, nitrogen, oxygen, sulfur, phosphorus, and halogens. The heteroalkyl group can be unsubstituted or substituted with one or more substituent. Examples of substituents include, but are not limited to, various substituents such as, for example, halogens (—F, —Cl, —Br, and —I), aliphatic groups (e.g., alkenes, alkynes), aryl groups, alkoxides, carboxylates, carboxylic acids, ether groups, and the like, and combinations thereof.

This disclosure provides compounds (e.g., water soluble bi-functional agents) for tumor imaging (e.g., magnetic resonance (MR) and fluorescence) that may be used in photodynamic therapy (PDT) methods. For example, 3-(1-hexyloxyethyl)pyropheophorbide-a (HPPH or Photochlor), a tumor-avid chlorophyll-a derivative currently undergoing human clinical trials, was conjugated with mono, di and tri Gd(DTPA) moieties, respectively. The T1/T2 relaxivity (in vitro/in vivo) and in vitro PDT efficacy of these conjugates were determined. The tumor-specificity of certain conjugates was also investigated at various time points in mice bearing Colon26 tumors and rabbits bearing widespread metastases from VX2 systemic arterial disseminated metastases. All the synthetic conjugates showed significant T₁ and T₂ relaxivities. As a bi-functional agent, the conjugate containing 3 Gd(III)-aminoethylamido-DOTA at position-17 of HPPH [HPPH-17-DOTA-Gd(III)] exhibited desirable properties, demonstrating great potential for tumor imaging by both MR and fluorescence while maintaining its PDT efficacy. MR imaging with HPPH-3GD(DOTA) successful identified minute (1.5 mm) VX2 metastases in rabbits, at 6 and 24 h after intravenous injection. At an MR imaging dose (10 μmole/kg) which is 10-fold lower than the Magnavist, the Gd HPPH-3Gd (DOTA) chelate did not produce any organ toxicity in mice. These results illustrate that compounds of the present disclosure (e.g., water soluble HPPH-3Gd(III) DOTA compounds) can be clinically suitable candidates, for example, a imaging or dual-imaging and therapeutic agent.

3(Gd)-DOTA-HPPH is an MRI agent, one that accumulates progressively in tumors for 24-48 hours, a potential replacement for F-18-FDG, so that total body MRI, enhanced with such a contrast medium, could replace both F-18-FDG-PET/MRI, and, incidentally, F-18-FDG-PET/CT. And: unlike the currently-available MRI tumor-enhancing agents, it remains on tumors for at least 24 hours, so that even at the end of a total body MRI there would be no “conspicuity fade” of the malignant lesion; this has never been achieved before with any other tumor-avid gadolinium-based contrast agent.

In an aspect, the present disclosure provides compounds. The compounds of the present disclosure can be made by methods disclosed herein. The compounds can be used as is described herein.

A compound comprises a tetrapyrrole (e.g., a tetrapyrrole group) linked via a linker to one or more ligands. In various examples, a compound comprises: F-L-R, where F is a tetrapyrrole (e.g., a porphyrin, such as, but not limited to, HPPH and derivatives and analogs thereof), optionally, comprising a chelating ion (e.g., deuterium (²H), ⁶⁴Cu, ¹¹¹In, ⁵⁷Ga), L is a linker, and R is a ligand, optionally, comprising a Gd(III) ion coordinated to the ligand.

A compound can comprise various tetrapyrroles (e.g., tetrapyrrole groups). A tetrapyrrole group may be derived from a tetrapyrrole. In an example, a tetrapyrrole (e.g., tetrapyrrole group) is tumor-avid tetrapyrrole. Non-limiting examples of tetrapyrroles (e.g., tetrapyrrole groups) include porphyrins, such as, but not limited to, HPPH and derivatives and analogs thereof, groups derived therefrom. In various examples, F is selected from the group consisting of:

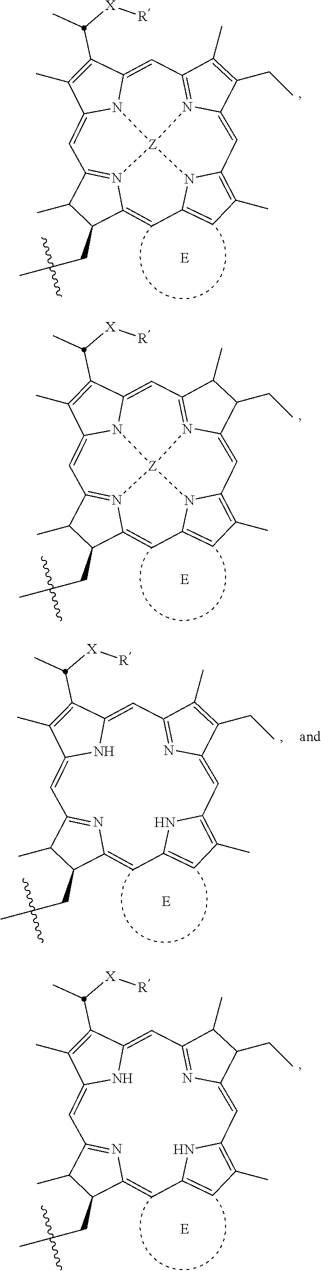

where R′ is an alkyl group or

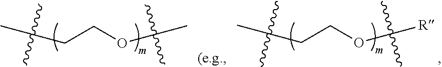

where R″ is an alkyl group, a peptide group, or a heteroalkyl group), where m is an integer from 1 to 12, including all integer values and ranges therebetween. X is selected from the group consisting of O, S, or NH. Z is selected from the group comprising ²H, ⁶⁴Cu, ¹¹¹In, and ⁵⁷Ga. E is selected from the group comprising (i) five-member isocyclic ring having a ketone or (ii) a six member N-substituted ring, where the N is functionalized with a substituent selected from the group consisting of an alkyl group, a heteroalkyl group, a peptide group (e.g., a linear peptide, a cyclic peptide, and derivatives or analogs thereof), or

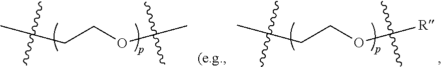

where R″ is an alkyl group, a peptide group, or heteroalkyl group), where p is an integer from 1 to 12, including all integer values and ranges therebetween. The dotted carbon is either chiral or achiral.

In various examples, E is:

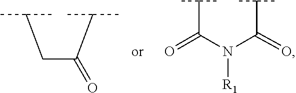

where R₁ is selected from the group consisting of an alkyl group, a heteroalkyl group, a peptide group (e.g., a linear peptide, a cyclic peptide, and derivatives or analogs thereof), or

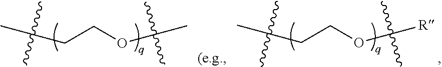

where R″ is an alkyl group, a peptide group, or a heteroalkyl group), where q is an integer from 1 to 12, including all integer values and ranges therebetween.

In various examples, L is selected from the group consisting of:

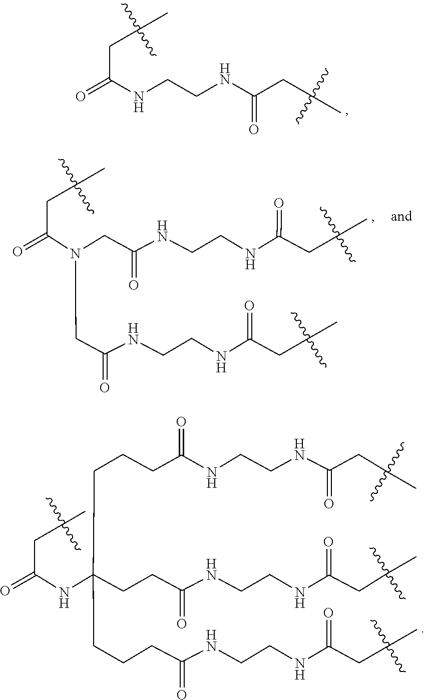 [SMILES: *CC(=O)NCCNC(=O)C* ; *CC(=O)NCCNC(=O)CN(CC(=O)NCCNC(=O)C*)C(=O)C* ; *CC(=O)NCCNC(=O)CCCC(CCCC(=O)NCCNC(=O)C*)(CCC(=O)NCCNC(=O)C*)NC(=O)C*]

In various examples, R is selected from the group consisting of:

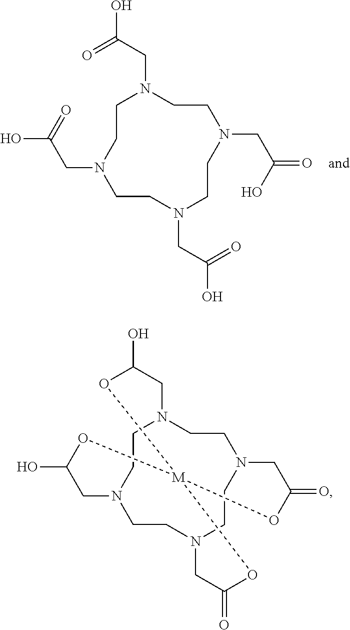

where M is a Gd(III) ion.

In various examples, the compound is:

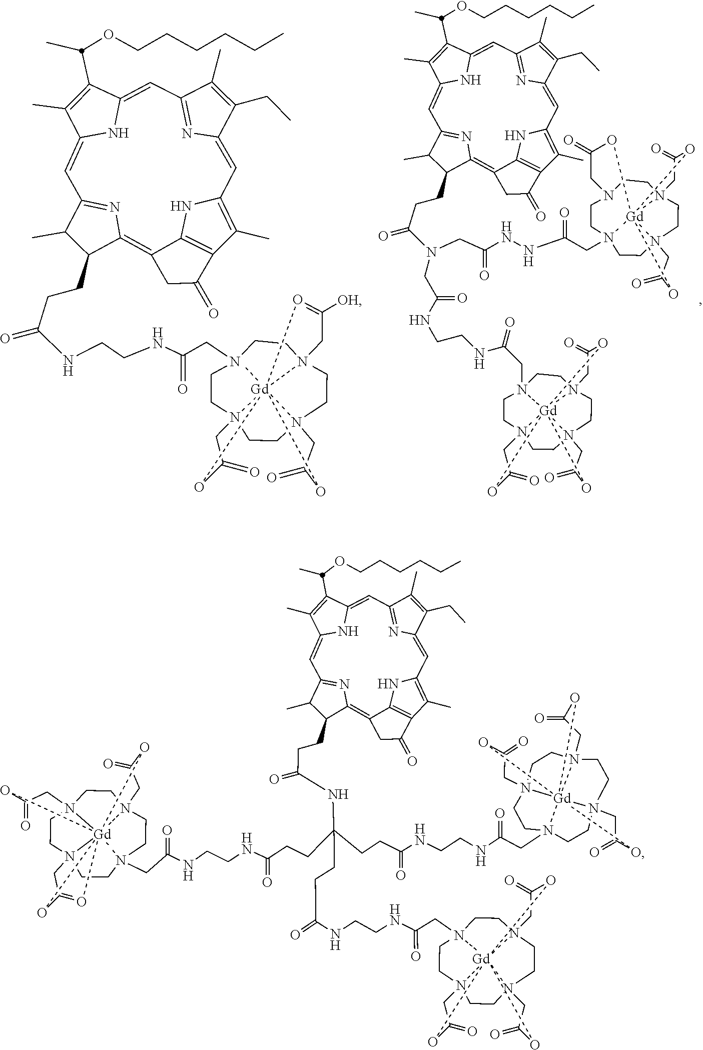

where the dotted carbon is either chiral or achiral.

In various examples, the compound is a salt, a partial salt, a hydrate, a polymorph, an isomer (e.g., a structural or stereoisomer), or a mixture thereof. The compounds can have stereoisomers. For example, the compound can be present as a racemic mixture, a single enantiomer, a single diastereomer, mixture of enantiomers, or mixture of diastereomers.

The compounds of the present disclosure include pharmaceutically acceptable derivatives and prodrugs of the compounds of the present disclosure. A compound may be a lyophilized compound (e.g., a lyophilized powder).

In an aspect, the present disclosure provides compositions comprising one or more compound of the present disclosure. The compositions may comprise one or more pharmaceutically acceptable carrier.

The compositions can include one or more standard pharmaceutically acceptable carriers. The compositions can include solutions, suspensions, emulsions, and solid injectable compositions that are dissolved or suspended in a solvent before use. The injections can be prepared by dissolving, suspending or emulsifying one or more of the active ingredients in a diluent. Examples of diluents are distilled water for injection, physiological saline, vegetable oil, alcohol, and a combination thereof. Further, the injections can contain stabilizers, solubilizers, suspending agents, emulsifiers, soothing agents, buffers, preservatives, etc. The injections, are sterilized in the final formulation step or prepared by sterile procedure. The pharmaceutical composition of the invention can also be formulated into a sterile solid preparation, for example, by freeze-drying, and can be used after sterilized or dissolved in sterile injectable water or other sterile diluent(s) immediately before use. Pharmaceutically-acceptable carriers include, but are not limited to, sugars, such as lactose, glucose and sucrose; starches, such as corn starch and potato starch; cellulose, including sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The composition, if desired, can also contain minor amounts of wetting or emulsifying agents, or pH buffering agents. Additional non-limiting examples of pharmaceutically acceptable carriers can be found in: Remington: The Science and Practice of Pharmacy (2005) 21st Edition, Philadelphia, Pa. Lippincott Williams & Wilkins.

In an aspect, the present disclosure provides uses of compounds of the present disclosure. The compounds can be used as imaging agents (e.g., MRI contrast agents) or as both imaging and therapeutic agents.

In various examples, the present disclosure provides methods that use one or more compounds of the present disclosure. Examples of methods include, but are not limited to, methods of imaging an individual (or a portion thereof) and methods of imaging and treating an individual.

Examples of uses of the compounds of the present disclosure include, but are not limited to:

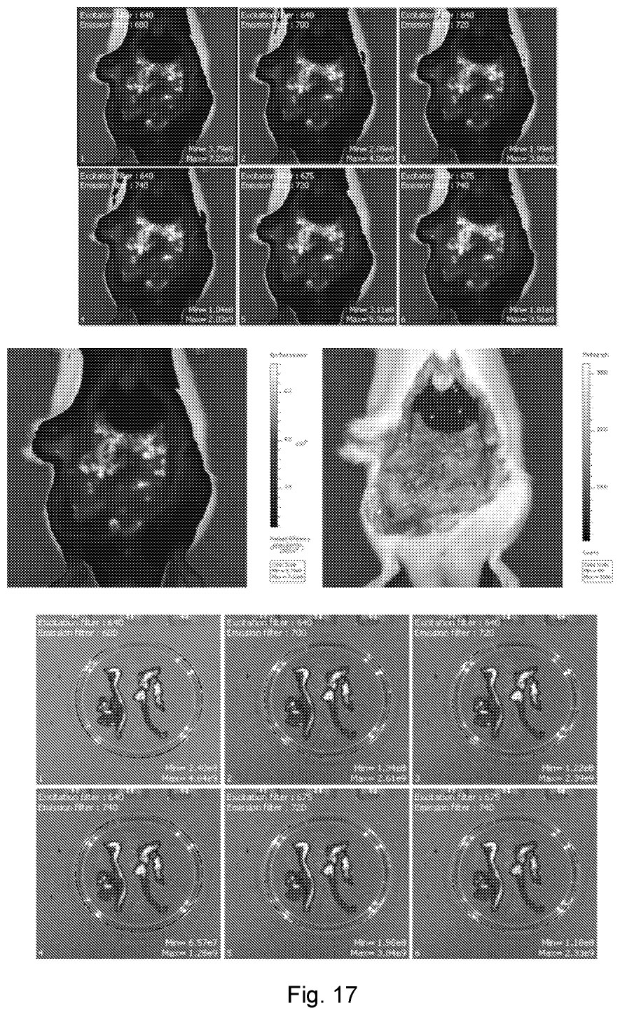

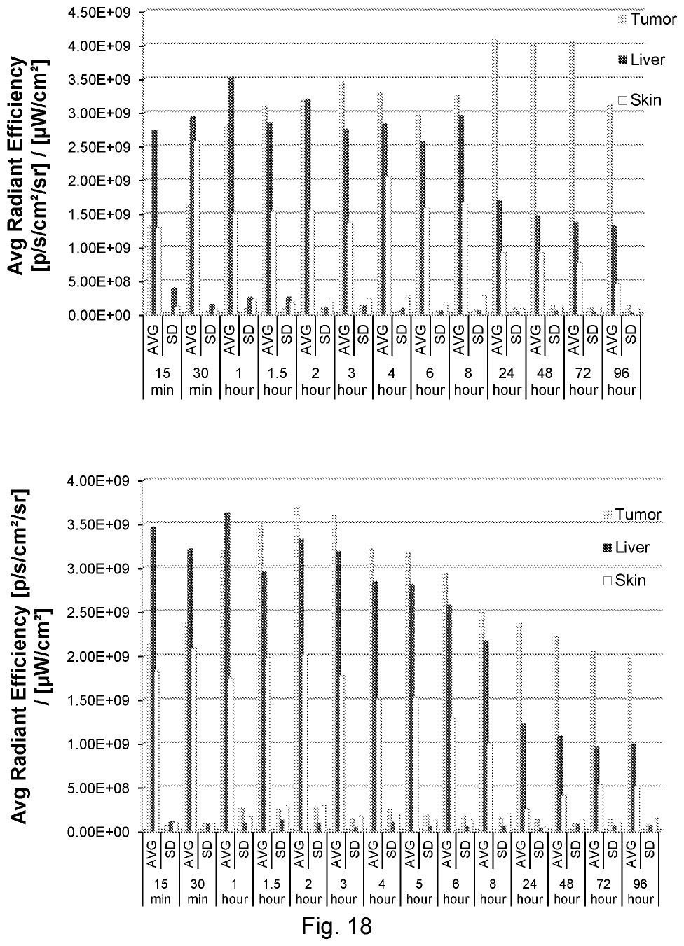

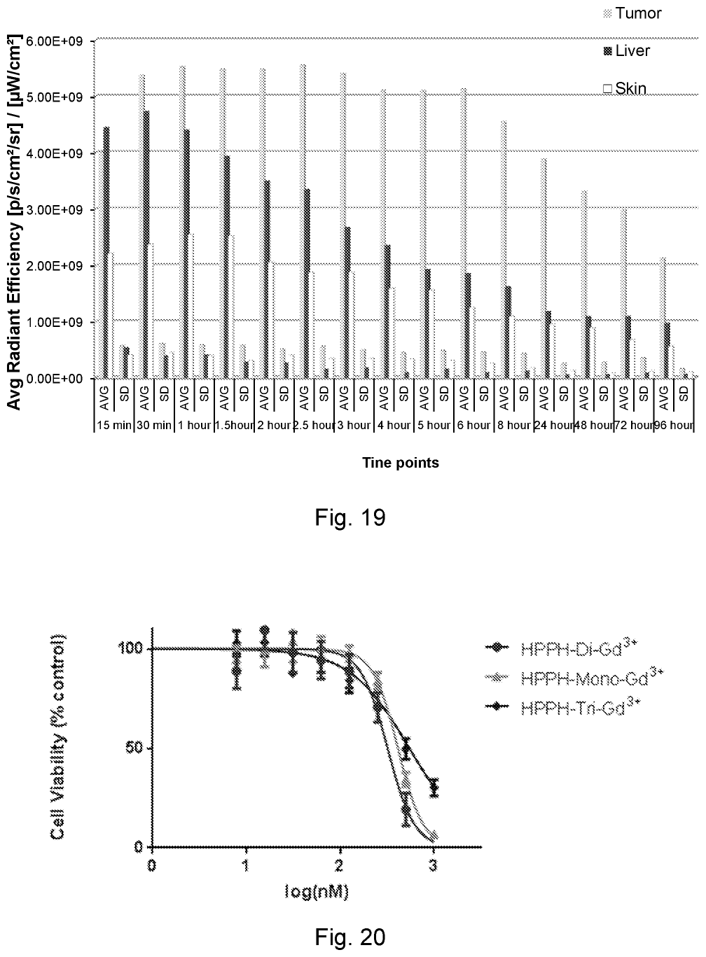

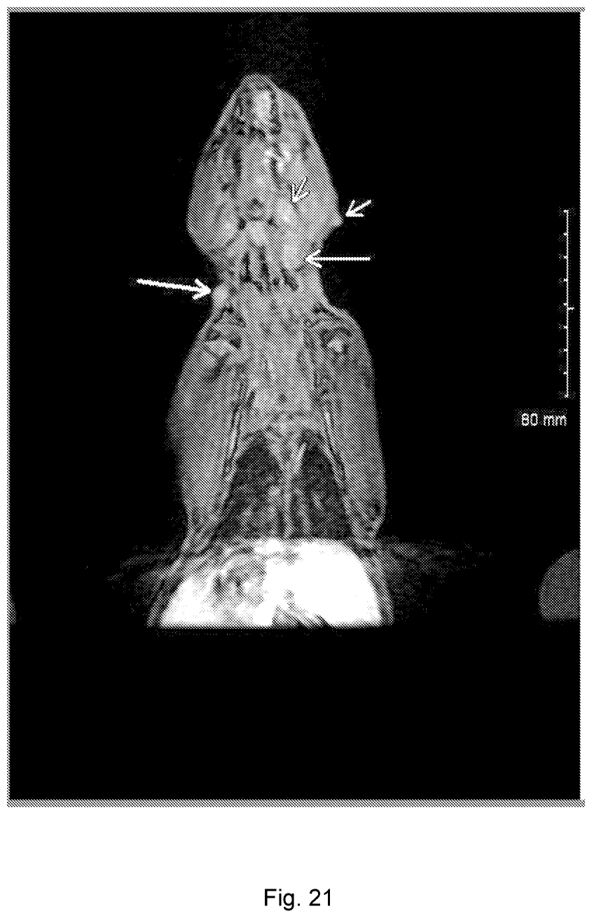

i) The compounds of the present disclosure were engineered to clear from the circulation slowly, such that hundreds of circulatory “passes” through, and by, tumor would be available for tumor binding. Thus, these molecules can also function as “blood pool agents,” analogous, for example, to albumin-binding gadolinium complexes.
ii) As demonstrated herein, compounds of the present disclosure (e.g., 3(Gd)-DOTA-HPPH complex), retains its fluorescence when exposed to light of the proper wavelength. Accordingly, based on this feature, intravenous injection of a compound (e.g., 3Gd-DOTA-HPPH), in patients with intraabdominal disease, for example, and then, at exploratory surgery (e.g., exploratory surgery occurring the next day), exposing the open abdomen to the correct wavelength of light for identification, and even treatment with photodynamic therapy (see, e.g., FIGS. 17 and 20).
iii) HPPH-Gd(DOTA) analogs can also be used as photosensitizers for the use in photodynamic therapy of cancer.
iv) Total-body MRI for tumor staging, performed, for example, 24 hr, after peripheral intravenous injection of a compound of the present disclosure (e.g., 3Gd(DOTA)-HPPH) would have all the advantages of F-18-FDG-PET/MRI, while delivering no radiation and at much lower cost. Only a standard MRI scanner would be required, obviating the high cost and space requirements of hybrid PET/MRI machines. Moreover, the compound (e.g., 3Gd(DOTA)-HPPH) may be useful for other ancillary purposes.
v) The compounds of the present disclosure (e.g., 3Gd(DOTA)-HPPH) are MRI contrast media that is truly tumor-avid and accumulates progressively in (or on) tumors, for example, for about 24 h; moreover, no previous Gd-based contrast medium has defined tumors so small, some no more than 1.5 mm in diameter, in animals of this size, at 1.5 Tesla. Additionally, the compounds exhibit a lack of canine toxicity (in the solvent it requires) and water solubility, so that it can be reconstituted on site.
vi) Conspicuity of a lesion would not fade for 24 h, so if a lesion is observed at 6 hours, it could be biopsied under MRI guidance the next day, without the need for another dose of contrast medium.
vii) Because non-lactating human breasts are 50-75% fat, minute breast tumors would theoretically be highly conspicuous against a fat-suppressed background. Breast-only MRI with compounds of the present disclosure is therefore an exciting possibility.
viii) Rabbits in examples described herein were imaged at 1.5 Tesla, whereas human trials would be at 3.0 Tesla; therefore, conspicuity of human lesions would be expected to be much greater. Moreover, please note that these images were created on a 26-year-old GE 1.5 T magnet, and, on a dedicated animal imaging system would have been FAR superior.

This disclosure provides methods of treating individuals in need of treatment (e.g., for a hyperproliferative disorder, such as, for example, malignancy (e.g., a malignancy disorder)) comprising administering to an individual a compound or composition of the present disclosure, and imaging the individual or a portion thereof and, after staging the disease, proceeding to appropriate therapy (e.g., surgical, chemotherapeutic, photodynamic, or standard radiation).

The image can be obtained using a techniques known in the art, such as, but not limited to, magnetic resonance imaging, and in some cases fluorescent imaging.

Methods of the present disclosure can be carried out in an individual who has been diagnosed with or is suspected of having cancer. A method can also be carried out in individuals who have a relapse or a high risk of relapse after being treated for cancer.

In various examples, a method for detecting the presence of a hyperproliferative tissue in an individual comprising: administering to the individual an effective quantity of one or more compound and/or one or more composition of the present disclosure; and imaging the individual or a portion thereof to detect the presence or absence of a hyperproliferative tissue in an individual.

A method may further comprise exposing the individual with light of a wavelength effective to treat the individual (e.g., kill or impair the hyperproliferative tissue).

The compound(s) and/or composition(s) may selectively interact(s) with hyperproliferative tissue relative to normal tissue, and a method may further comprise exposing (e.g., irradiating) the individual with light of a wavelength to kill or impair the hyperproliferative tissue. A method may also further comprise allowing time for any of the compound(s) that is/are not selectively interacted with the hyperproliferative tissue to clear from the normal tissue of the subject prior to the step of exposure (e.g., irradiation).

A method may use one or more lyophilized compound and/or one or more composition comprising one or more lyophilized compound. The lyophilized compound(s) and/or composition(s) may be reconstituted prior (e.g., immediately prior) to administration to the individual and immediately prior to imaging.

Methods of the present disclosure can be used on various individuals. Individuals are also referred to herein as subjects. In various examples, an individual is a human or non-human mammal. Examples of non-human mammals include, but are not limited to, farm animals, such as cows, hogs, sheep, and the like, as well as pet or sport animals such as horses, dogs, cats, and the like. Additional non-limiting examples of individuals include rabbits, rats, and mice.

“Hyperproliferative disorders” as used herein denotes those conditions disorders sharing as an underlying pathology excessive cell proliferation caused by unregulated or abnormal cell growth, and include uncontrolled angiogenesis. Examples of such hyperproliferative disorders includes, but are not limited to, cancers or carcinomas.

Various hyperproliferative tissues can be imaged and/or treated using methods of the present disclosure. Non-limiting examples of hyperproliferative tissues include vascular endothelial tissue, a neovasculature tissue, a neovasculature tissue present in the eye, an abnormal vascular wall of a tumor, a solid tumor, a tumor of a head, a tumor of a neck, a tumor of an eye, a tumor of a gastrointestinal tract, a tumor of a liver, a tumor of a breast, a tumor of a prostate, a tumor of a lung, a nonsolid tumor, malignant cells of one of a hematopoietic tissue and a lymphoid tissue. Combinations of hyperproliferative tissues can be imaged and/or treated.

In various examples, a method of the present disclosure comprises administering to an individual one or more compound and/or one or more composition of the present disclosure. The compound(s) and/or composition(s) can be delivered to the vascular system of an individual such as, by using intravascular delivery.

The compounds can act as conventional PDT agents. PDT methods are known in the art. The compounds of the present disclosure can be used in dual mode methods. In such methods, the compounds act as both a contrast agent/medium and as a therapeutic agent (e.g. as a photodynamic therapy (PDT) agent). Accordingly, dual mode methods comprise both an imaging step and an exposure (e.g., irradiation) step: the “see and treat” algorithm.

“Irradiating” and “irradiation” as used herein includes exposing an individual to all wavelengths of light. Preferably, the irradiating wavelength is selected to match the wavelength(s) which excite the photosensitizing compound. Preferably, the radiation wavelength matches the excitation wavelength of the photosensitizing compound and has low absorption by the non-target tissues of the individual, including blood proteins, because the non-target tissues have not absorbed the PDT compound. In an example, a subject is exposed (e.g., irradiated) to a wavelength of light to kill or impair the hyperproliferative tissue through, for example, excitation of a compound and/or composition of the present disclosure.

Exposure (e.g., irradiation) is further defined herein by its coherence (laser) or non-coherence (non-laser), as well as intensity, duration, and timing with respect to dosing using the photosensitizing compound. The intensity or fluence rate must be sufficient for the light to reach the target tissue. The duration or total fluence dose must be sufficient to photoactivate enough photosensitizing compound to act on the target tissue. Timing with respect to dosing with the photosensitizing compound is important, because 1) the administered photosensitizing compound requires some time to home in on target tissue and 2) the blood level of many photosensitizing compounds decreases with time. The radiation energy is provided by an energy source, such as a laser or cold cathode light source, that is external to the individual, or that is implanted in the individual, or that is introduced into an individual, such as by a catheter, optical fiber or by ingesting the light source in capsule or pill form (e.g., as disclosed in. U.S. Pat. No. 6,273,904 (2001)).

In an example, a method is carried out using a single MRI scanner and a single dose of the compound(s) and/or composition(s). In another example; a method is carried out using a single dose of the compound(s) and/or composition(s). In another example; a method is carried out using a single dose of the compound(s) and/or composition(s) and no radiation exposure.

In various examples, other forms of energy (e.g., forms of energy other than light energy during administering and excitation of a PDT compound and/or composition to treat an individual) are within the scope of this disclosure to treat an individual (e.g., to kill or impair the hyperproliferative tissue), as will be understood by those of ordinary skill in the art.

As used herein, destroy means to kill the desired target tissue or target composition, including infecting agents. “Impair” means to change the target tissue or target composition in such a way as to interfere with its function or reduce its growth. For example, in North et al., it is observed that after virus-infected T cells treated with benzoporphyrin derivatives were exposed to light, holes developed in the T cell membrane and increased in size until the membrane completely decomposed (Blood Cells 18: 129-40 (1992)). The target tissue or target composition is understood to be impaired or destroyed even if the target tissue or target composition is ultimately disposed of by macrophages.

In an aspect, the present disclosure provides products, (e.g., articles of manufacture, such as, for example, kits), which comprise pharmaceutical preparations containing any one or any combination of the compounds described herein. In an example, the instant disclosure includes a closed or sealed package that contains the pharmaceutical preparation. In certain examples, the package can comprise one or more closed or sealed vials, bottles, blister (bubble) packs, or any other suitable packaging for the sale, or distribution, or use of the pharmaceutical compounds and compositions comprising them. The printed material can include printed information. The printed information can be provided on a label, or on a paper insert, or printed on the packaging material itself. The printed information can include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material can include an indication that the pharmaceutical composition and/or any other agent provided with it is for treatment of cancer and/or any disorder associated with cancer. In examples, the product includes a label describing the contents of the container and providing indications and/or instructions regarding use of the contents of the container to treat any cancer.

The steps of the methods described in the various embodiments and examples disclosed herein are sufficient to carry out the methods of the present disclosure. Thus, in an example, a method consists essentially of a combination of the steps of the methods disclosed herein. In another example, a method consists of such steps.

The following Statements are examples of compounds of the present disclosure and examples of uses of same:

Statement 1. A compound comprising:

### F-L-R,

where F is a tetrapyrrole group (e.g., a porphyrin group, such as, but not limited to, HPPH and derivatives and analogs thereof), optionally, comprising a chelating ion (e.g., deuterium (2H), ⁶⁴Cu, ¹¹¹In, ⁵⁷Ga), L is a linker, and R is a ligand, optionally, comprising a Gd(III) ion coordinated to the ligand.
Statement 2. The compound according to Statement 1, where F is chosen from

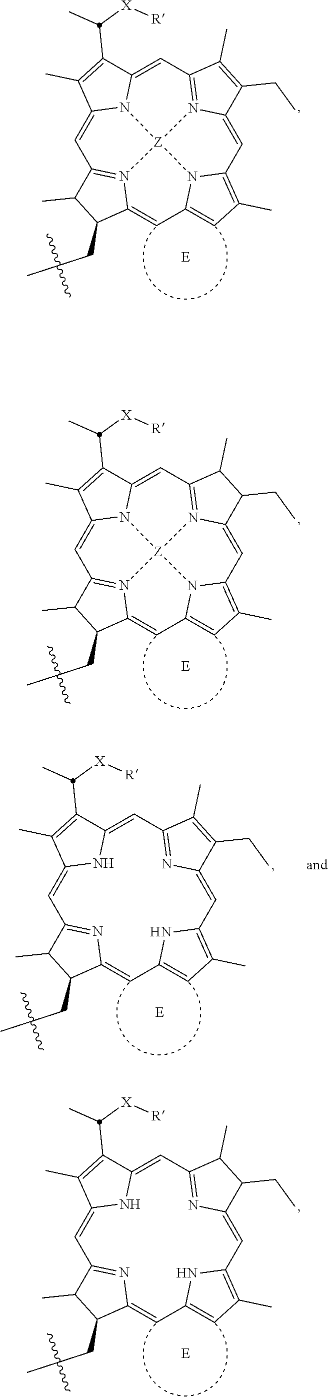

where R′ is an alkyl group or

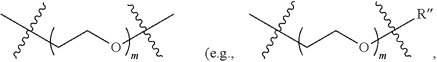

where R″ is chosen from an alkyl group, a peptide group, and a heteroalkyl group), where m is an integer from 1 to 12, including all integer values and ranges therebetween;
X is chosen from O, S, and NH;
Z is chosen from ²H, ⁶⁴Cu, ¹¹¹In, and ⁵⁷Ga;
E is (i) a five-member isocyclic ring having a ketone or (ii) a six member N-substituted ring, where the N is functionalized with a substituent selected from the group consisting of an alkyl group, a heteroalkyl group, a peptide group (e.g., a linear peptide, a cyclic peptide, and derivatives or analogs thereof), or

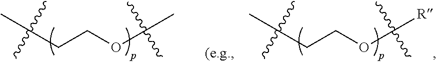

where R″ is chosen from an alkyl group, a peptide group, and a heteroalkyl group), where p is an integer from 1 to 12, including all integer values and ranges therebetween;
R″ is at each occurrence independently chosen from an alkyl group, a peptide group, and a heteroalkyl group and
the dotted carbon is either chiral or achiral.
Statement 3. The compound according to Statement 2, where E is:

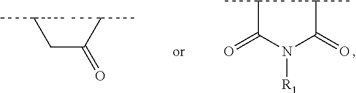

where R₁ is selected from the group consisting of an alkyl group, a heteroalkyl group, a peptide group (e.g., a linear peptide, a cyclic peptide, and derivatives or analogs thereof), or

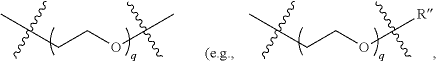

where R″ is chosen from an alkyl group, a peptide group, and a heteroalkyl group), where q is an integer from 1 to 12, including all integer values and ranges therebetween.
Statement 4. The compound of any of the preceding Statements, where L is chosen from:

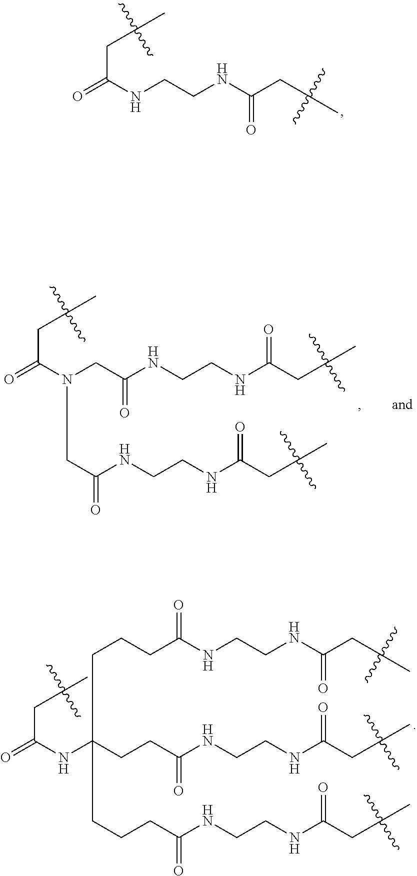 [SMILES: *CC(=O)NCCNC(=O)C* ; *CC(=O)NCCNC(=O)CN(CC(=O)NCCNC(=O)C*)C(=O)C* ; *CC(=O)NCCNC(=O)CCCC(CCCC(=O)NCCNC(=O)C*)(CCC(=O)NCCNC(=O)C*)NC(=O)C*]

Statement 5. The compound of any of the preceding Statements, where R is chosen from:

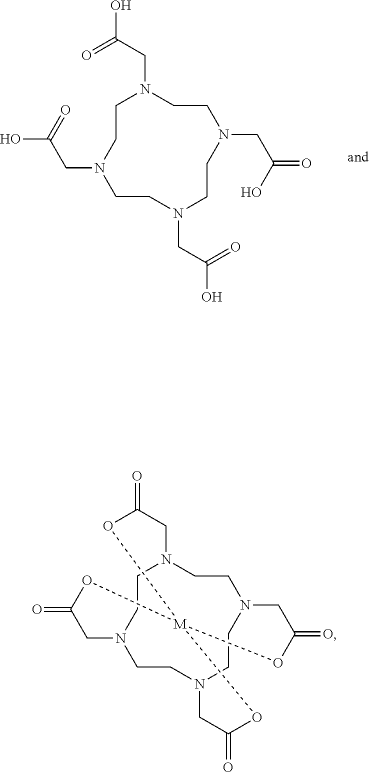

where M is a Gd ion (e.g., Gd(III) ion).
Statement 6. The compound of any of the preceding Statements, where the compound is:

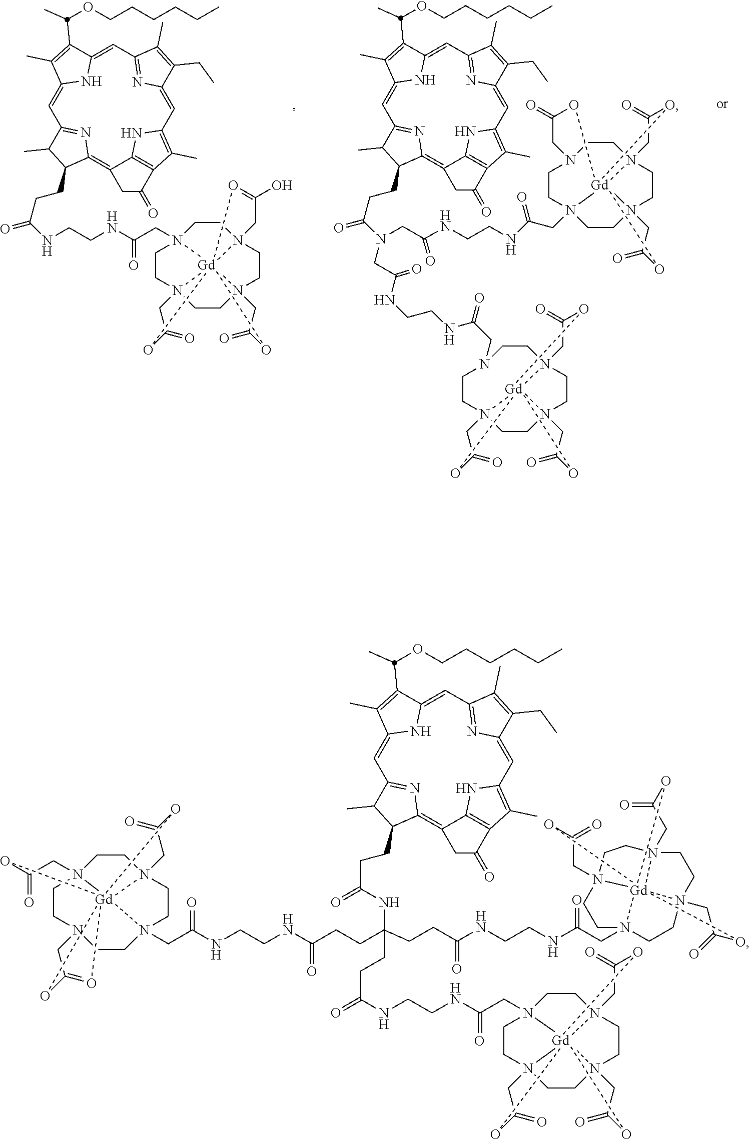

where the dotted carbon is either chiral or achiral.
Statement 7. A composition comprising one or more compound of the present disclosure (e.g., one or more compound of any of the preceding Statements).
Statement 8. The composition according to Statement 7, where the composition further comprises a pharmaceutically acceptable carrier.
Statement 9. A method for detecting the presence of a hyperproliferative tissue in an individual comprising:

administering to the individual an effective quantity of one or more compound and/or one or more composition of the present disclosure (e.g., one or more compound of any one or more of Statements 1-6 and/or one or more compositions of any one of Statements 7-8); and

imaging the individual or a portion thereof to detect the presence or absence of a hyperproliferative tissue (e.g., the presence of a hyperproliferative issue containing a compound and/or composition of the present disclosure) in an individual.

Statement 10. The method according to Statement 9, where the method further comprises:

exposing (e.g., irradiating) the individual with light of a wavelength to kill or impair the hyperproliferative tissue.

Statement 11. The method according to Statement 10, where the compound(s) and/or composition(s) selectively interact(s) with hyperproliferative tissue relative to normal tissue, and exposing (e.g., irradiating) the subject with light of a wavelength to kill or impair the hyperproliferative tissue.
Statement 12. The method according to Statement 11, further comprising allowing time for any of the compound(s) that is/are not selectively interacted with the hyperproliferative tissue to clear from the normal tissue of the subject prior to the step of exposing.
Statement 13. A method of photodynamic therapy for treating hyperproliferative tissue in an individual, comprising:

(i) administering to the individual one or more compound and/or one or more composition of the present disclosure (e.g., one or more compound of any one or more of Statements 1-6 and/or one or more compositions of any one of Statements 7-8) that selectively interacts with the hyperproliferative tissue relative to normal tissue, and

(ii) exposing (e.g., irradiating) the individual with light of a wavelength to activate (e.g., excite) the compound, whereby the hyperproliferative tissue is treated (e.g., destroyed and/or impaired).

Statement 14. The method of any one according to any of Statements 9-13, where the imaging is magnetic resonance (MR) imaging and/or fluorescence imaging.
Statement 15. The method of any one according to any of Statements 9-14, where the hyperproliferative tissue is a vascular endothelial tissue, a neovasculature tissue, a neovasculature tissue present in the eye, an abnormal vascular wall of a tumor, a solid tumor, a tumor of a head, a tumor of a neck, a tumor of an eye, a tumor of a gastrointestinal tract, a tumor of a liver, a tumor of a breast, a tumor of a prostate, a tumor of a lung, a nonsolid tumor, and malignant cells of one of a hematopoietic tissue and a lymphoid tissue.
Statement 16. The method of any one according to any of Statements 9-15, where the administering comprises reconstituting one or more compound and/or one or more composition of the present disclosure (e.g., one or more compound of any one or more of Statements 1-6 and/or one or more compositions of any of Statements 7-8); and administering the reconstituted one or more compound(s) and/or one or more composition(s) to the individual.
Statement 17. The method according to Statement 16, where the one or more compound is one or more lyophilized compound of the present disclosure (e.g., one or more compound of any one or more of Statements 1-6) and/or one or more composition comprises one or more lyophilized compound of the present disclosure (one or more compound of any one or more of Statements 1-6).
Statement 18. A kit comprising

one of more compound of any of Statements 1-6 and/or one or more composition of any of Statements 7-8; and

instructions for use of the one or more compound and/or the one or more composition.

The following examples are presented to illustrate the present disclosure. They are not intended to limiting in any matter.

### EXAMPLE 1

This example provides a description of the compounds of the present disclosure, methods of making compounds of the present disclosure, and uses of compounds of the present disclosure.

For the synthesis of desired conjugates HPPH was prepared from chlorophyll-a by following the methodology. It was converted to the corresponding mono- di- and tri or 3-Gd DOTA conjugates by following the methodology depicted in Schemes 1-3.

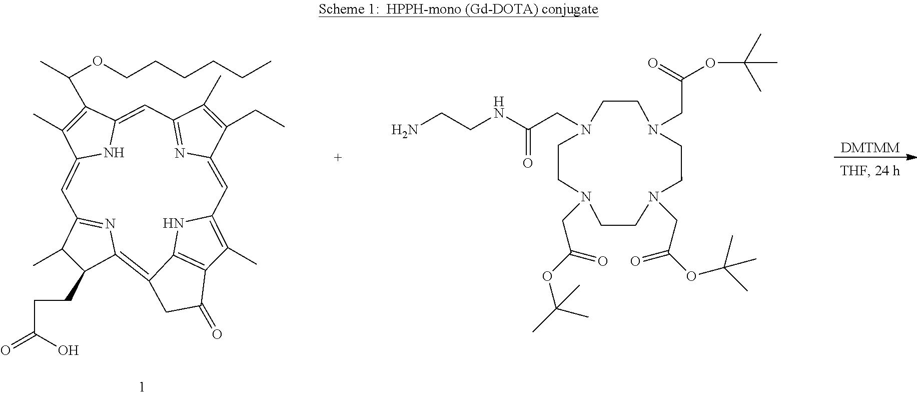

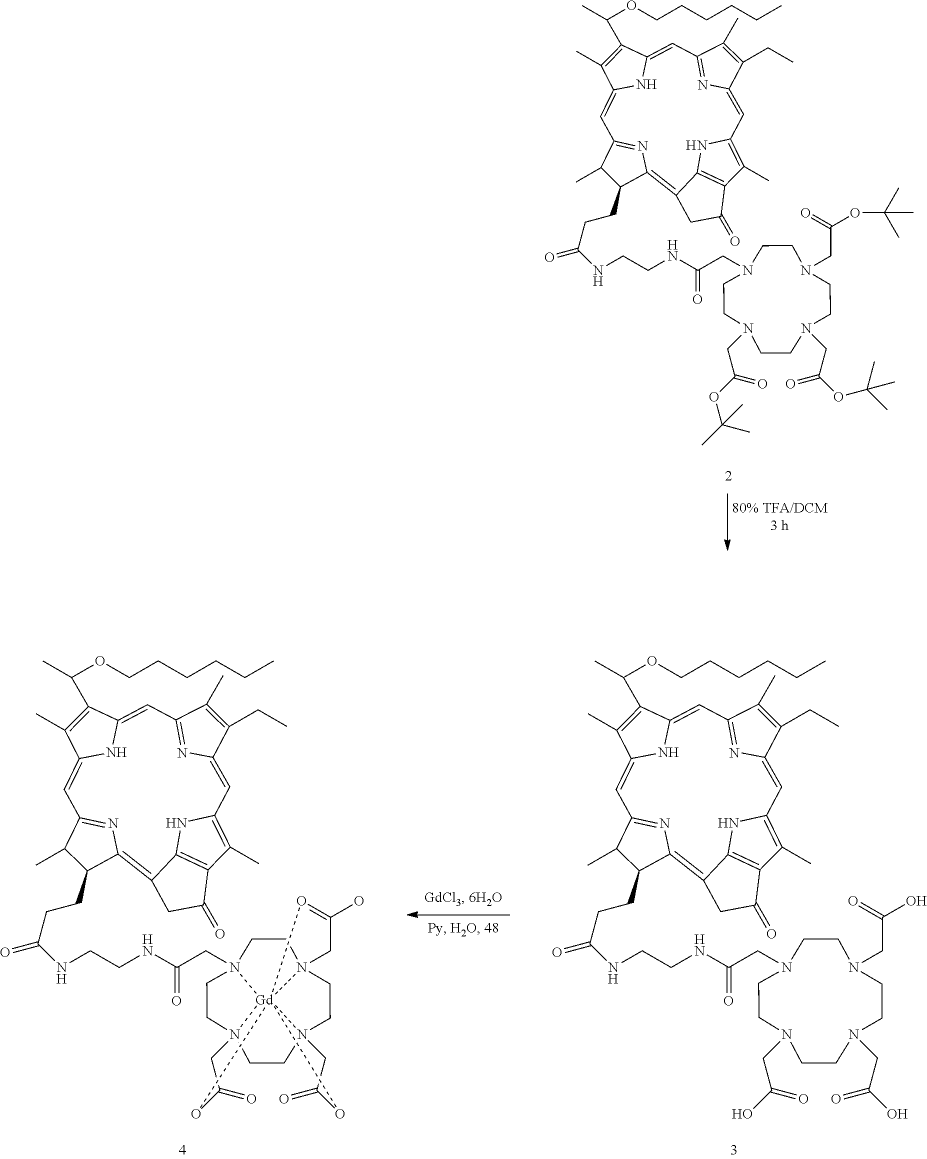

Synthesis of compound 2: A mixture of HPPH (400 mg, 0.628 mmol), 2-aminoethyl-DOTA-tris(t-Bu ester) (655.2 mg, 0.802 mmol), 4-(4,6-di methoxy-1,3,5,-triazin-2yl)-4-methyl morpholinium chloride (DMTMM) (208.5 mg, 0.754 mmol) in dry THF (30 mL) was stirred at room temperature under argon for 24 h. Water (3 mL) was added, and the reaction mixture was allowed to stir for 15 min. The organic layer was separated, dried with sodium sulfate, filtered and concentrated. The residue was purified with column chromatography using Alumina grade III using 3% methanol/DCM mixture as eluent to give product 2 with 81% yield (627.8 mg). ¹H NMR (400 MHz, CDCl₃, δ ppm): 9.74/9.73 (1H, s, 5-H), 9.48 (1H, s, 10-H), 9.03 (1H, br m, —NHCH₂CH₂NH—), 8.79 (1H, br t, J˜5 Hz —NHCH₂CH₂NH—), 8.62/8.61 (1H, s, 20-H), 5.90/5.89 (1H, q, J=6.8 Hz, 3¹-H), 5.37 (1H, d, J=20.2 Hz, 13¹-CHH), 5.11 (1H, d, J=20.2 Hz, 13¹-CHH), 4.73 (1H, q, J=7.3 Hz, 18-H), 4.27 (1H, d, J=10.4 Hz, 17-H), 3.70 (2H, q, J=7.7 Hz, 8-CH₂CH₃), 3.54-3.69 (2H, m, —OCH₂(CH₂)₄CH₃), 3.66 (3H, s, 12-CH₃), 1.0-3.6 (24H, br m, DOTA ring and exocyclic CH₂ groups, see notes below), 3.41 (2H, br s, —NHCH₂CH₂NH—), 3.378/3.376 (3H, s, 2-CH₃), 3.35 (2H, br m, —NHCH₂CH₂NH—), 3.25 (3H, s, 7-CH₃), 2.84 (1H, m, 1H of 17-CH₂CH₂—), 2.80 (1H, m, 1H of 17-CH₂CH₂—), 2.60 (1H, m, 1H of 17-CH₂CH₂—), 2.11/2.10 (3H, d, J=6.7 Hz, 3¹-CH₃), 2.07 (1H, m, 1H of 17-CH₂CH₂—), 1.81 (3H, d, J=7.3 Hz, 18-CH₃), ˜1.73 (2H, m, —OCH₂CH₂(CH₂)₃CH₃), 1.70 (3H, t, J=7.6 Hz, 8-CH₂CH₃), 1.40, 1.39, 1.38 (18H, 3 x s, 2 x —COOC(CH₃)₃), 1.34 (9H, br s, —COOC(CH₃)₃), 1.29-1.46 (2H, m, —O(CH₂)₂CH₂(CH₂)₂CH₃), 1.16-1.27 (4H, m, —O(CH₂)3(CH₂)₂CH₃), 0.77 (3H, distorted t, J˜7 Hz, —O(CH₂)₅CH₃), 0.41/0.40 (1H, s, core NH), −1.74/−1.75 (1H, s, core NH); ¹³C NMR (100 MHz, CDCl₃, δ ppm): 196.53/196.52, 173.43/173.42, 172.76/172.75, 172.1, 171.9, 171.9, 171.4, 162.18/162.15, 154.9, 150.5, 149.1, 144.6, 141.3/141.2, 139.31/139.26, 137.5, 136.0, 135.6/135.5, 132.7/132.6, 130.5, 127.78/127.77, 106.29/106.27, 103.6, 97.4, 93.6/93.5, 82.0, 81.94, 81.91, 72.82/72.79, 69.6, 56.0, 55.54, 55.48, 55.4, 47-53 (see notes below), 52.3, 49.8, 48.2, 39.3, 39.13/39.09, 33.61/33.59, 31.7, 31.2, 30.2, 28.0, 27.92, 27.87, 26.0, 24.8/24.7, 23.5, 22.6/22.5, 19.5, 17.5, 14.0, 12.0, 11.3, 11.1. MS (ESI) m/z: 1233.80 (M+H+). UV-vis (MeOH, λmax, nm (ε): 660 (0.645), 604 (0.116), 536.9 (0.127), 504 (0.123), 408 (1.260), 319 (0.306).

Synthesis of compound 3: (500 mg, 0.4055 mmol) was stirred with 80% trifluoroacetic acid (TFA)/dichloromethane (DCM, 50 mL) at room temperature for 3 h. The resultant mixture was concentrated and the pH was adjusted to 6.5 by adding sat. NaHCO₃ dropwise. The crude reaction mixture was then diluted with dichloromethane (100 mL), washed with water (3×100 mL), dried over anhydrous sodium sulfate and concentrated down to yield product 3 with 91% yield (392.82 mg). MS (ESI) m/z: 1063.59 (M−H⁺). HRMS (ESI): calcd for C₅₇H₈₀N₁₀O₁₀ (M⁺) 1064.6011; found, 1064.6028. UV-vis (MeOH, λmax, nm (ε): 660 (0.810), 604 (0.156), 536.9 (0.164), 505.1 (0.159), 408 (1.686), 319 (0.424).

Synthesis of compound 4: Compound 3 (350 mg, 0.329 mmol) was dissolved in pyridine (35 mL), and while stirring, GdCl₃.6H₂O (245 mg, 0.657 mmol) in 3.5 mL of DI water added slowly, and the resultant mixture was stirred at room temperature for 48 h. The reaction mixture was concentrated to dryness under high vacuum. The residue was washed with DCM (2×20 mL) and acetone (2×20 mL). The crude reaction mixture was purified by Bio-Gel P-6 (medium) column using DI water twice and 1K 50 mL centrifugal devices for 4 times (each tube 100 mg) as eluent to give product 4 with 86% yield (344.84 mg). MS (ESI) m/z: 1218.50 (M−H⁺). HRMS (ESI): calcd for C₅₇H₇₈N₁₀O₁₀Gd (M−H⁺) 1218.5001; found, 1218.5033. UV-vis (MeOH, λmax, nm (c)): 659 (3.96×10⁴), 604 (2.07×10⁵), 536 (1.93×10⁵), 505 (1.96×10⁵), 407 (1.87×10⁴), 316.9 (7.53×10⁴).

Synthesis of compound 5: HPPH (500 mg, 0.785 mmol), di-tert-butyl iminodiacetate (385 mg, 1.57 mmol), EDCI (301 mg, 1.57 mmol), and DMAP (191.8 mg, 1.57 mmol) were taken in a dry 500 mL round bottom flask. Dry dichloromethane (150 mL) was added and the reaction mixture was stirred at room temperature for 16 h under N₂ atm. The reaction mixture was diluted with dichloromethane (300 mL), washed with brine solution, the organic layer was separated, dried over anhydrous sodium sulfate, and concentrated. The crude reaction mixture was chromatographed over silica gel using 1-1.5% MeOH/dichloromethane mixture as eluent to give product 5 with 86% yield (583.48 mg).

Synthesis of compound 6: Compound 5 (400 mg, 0.4629 mmol) was stirred with 75% trifluoroacetic acid (TFA)/dry dichloromethane (DCM, 35 mL) at room temperature for 3 h. The resultant mixture was concentrated and the pH was adjusted to 6.5 by adding sat. NaHCO₃ dropwise and washed with DCM/MEOH (95:5) and DI water, organic layer was separated and dried over sodium sulfate, and concentrated down to yield crude product 6 with 90% yield (313.25 mg). UV-vis (MeOH, λmax, nm (c)): 660 (0.839), 604 (0.158), 536.9 (0.169), 505.1 (0.161), 408 (1.679), 319 (0.432).

Synthesis of Compound 7: Dry dichloromethane (80 mL) was added to compound 6 (300 mg, 0.399 mmol), 2-aminoethyl-DOTA-tris(t-Bu ester) (694 mg, 0.998 mmol), EDCI (305.94 mg, 1.596 mmol), and DMAP (195 mg, 1.596 mmol) in a 250 mL round bottom flask. The reaction mixture was stirred at room temperature for 24 h under argon atmosphere. After completion of the reaction, which was determined by TLC, the reaction mixture was diluted with DCM (100 mL), washed with brine solution, the organic layer was separated, dried over sodium sulfate, and concentrated down to yield crude product which was purified by Alumina, GRADE III using 2-4% methanol/DCM mixture as eluent to give product 7 with 79% yield (613.22 mg). ¹H NMR (400 MHz, CDCl₃, δ ppm): 9.83/9.76 (1H, s, 5-H), 9.49/9.48 (1H, s, 10-H), 9.41 (1H, br s, —NHCH₂CH₂NH—), 8.82 (1H, br t, J˜5 Hz, —NHCH₂CH₂NH—), 8.63 (1H, s, 20-H), 8.33 (1H, br s, —NHCH₂CH₂NH—), 7.87 (1H, distorted br s, —NHCH₂CH₂NH—), 5.90/5.87 (1H, q, J=6.7 Hz, 31-H), 5.250/5.246 (1H, d, J=20.1 Hz, 131-CHH), 5.11 (1H, d, J=20.1 Hz, 131-CHH), −1.4-4.7 (52H, br m, DOTA ring and exocyclic CH₂ groups & —N(CH₂C═ONH—)₂ of linker, see notes below), 4.54 (1H, q, J=7.2 Hz, 18-H), 4.21 (1H, d, J=10.4 Hz, 17-H), ˜3.68 (2H, m, 8-CH₂CH₃), 3.651/3.648 (3H, s, 12-CH₃), ˜3.63 (2H, m, —OCH₂(CH₂)₄CH₃), ˜3.3-3.6 (8H, br s, 2 x —NHCH₂CH₂NH—), 3.40/3.39 (3H, s, 2-CH₃), 3.24 (3H, s, 7-CH₃), 2.65, 2.86 (3H, 2 x brs m, 17-CHHCH₂—), 2.12/2.08 (3H, d, J=6.7 Hz, 31-CH₃), 1.93 (1H, br m, 17-CHHCH₂—), 1.79 (3H, d, J=7.2 Hz, 18-CH₃), ˜1.73 (2H, m, —OCH₂CH₂(CH₂)₃CH₃), 1.723/1.720 (3H, t, J=7.6 Hz, 8-CH₂CH₃), ˜1.45 (27H, m, 9 x t-butyl CH₃), 1.35-1.45 (2H, m, —O(CH₂)₂CH₂(CH₂)₂CH₃), 1.16-1.32 (31H, m, 9 x t-butyl CH₃ & —O(CH₂)₃(CH₂)₂CH₃), 0.84/0.75 (3H, distorted t, J˜7 Hz, —O(CH₂)₅CH₃), 0.33/0.31 (1H, s, core NH), −1.85/−1.87 (1H, s, core NH); ¹³C NMR (100 MHz, CDCl₃, δ ppm): 195.7, 174.2, 172.4, 172.09, 172.08, 172.02, 171.99, 171.96, 171.2/171.1, 169.3, 161.8/161.7, 155.03/155.01, 150.7/150.6, 148.80/148.79, 144.8, 141.4/141.2, 139.8/139.7, 137.51/137.49, 136.4/136.3, 135.6/135.4, 133.0/132.7, 130.63/130.60, 128.1/128.0, 106.34/106.29, 103.6, 97.9/97.7, 93.8, 81.82, 81.75, 81.71, 81.6, 81.5, 81.4, 72.8, 69.80/69.77, 56.3, 55.7, 55.3, 55.2, 45-54, 52.3, 50.0, 47.9, 39.4, 39.2, 38.1, 31.8/31.7, 31.2, 30.3/30.2, 28.08, 28.07, 27.96, 27.88, 27.82, 27.80, 27.77, 26.1/26.0, 25.2/24.6, 23.2, 22.6/22.5, 19.5, 17.70/17.68, 14.0/13.9, 12.0, 11.31/11.29, 11.2/11.1. MS (ESI) m/z: 1046.44 (M+H⁺). UV-vis (MeOH, λmax, nm (ε): 659 (0.476), 602 (0.083), 536 (0.095), 505.1 (0.963), 409.1 (0.963), 319 (0.209).

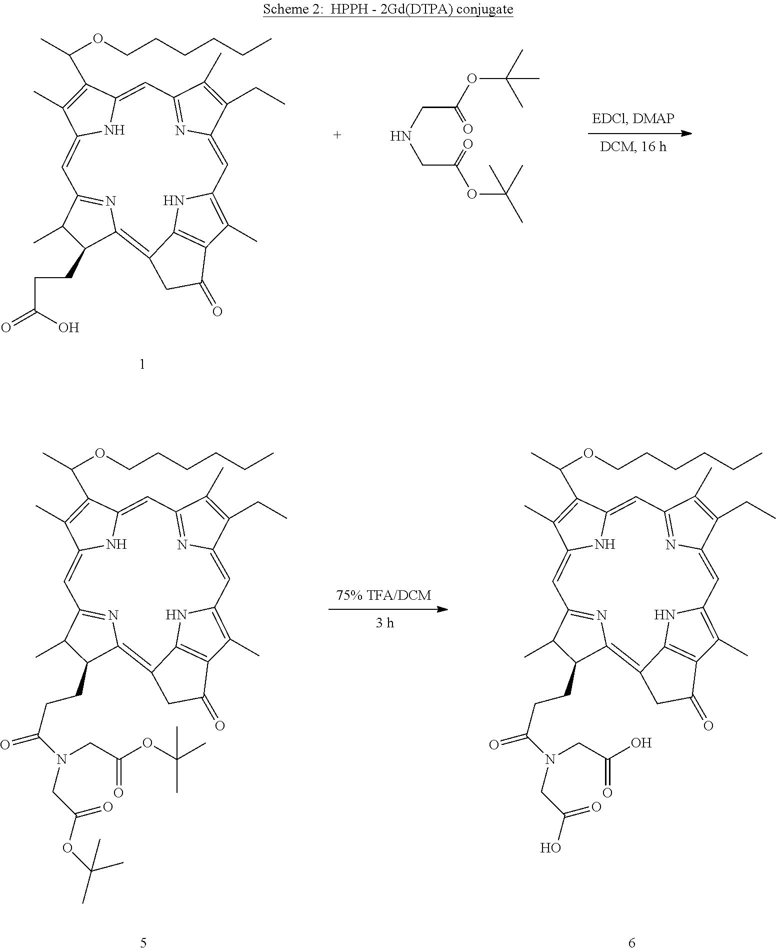 [SMILES: CCCCCCOC(C)c1c(C)c2cc3nc(c4c5[nH]c(cc6nc(cc1[nH]2)C(C)=C6CC)c(C)c5C(=O)C4)[C@@H](CCC(=O)O)C3C ; CC(C)(C)OC(=O)CNCC(=O)OC(C)(C)C ; CCCCCCOC(C)c1c(C)c2cc3nc(c4c5[nH]c(cc6nc(cc1[nH]2)C(C)=C6CC)c(C)c5C(=O)C4)[C@@H](CCC(=O)N(CC(=O)OC(C)(C)C)CC(=O)OC(C)(C)C)C3C ; CCCCCCOC(C)c1c(C)c2cc3nc(c4c5[nH]c(cc6nc(cc1[nH]2)C(C)=C6CC)c(C)c5C(=O)C4)[C@@H](CCC(=O)N(CC(=O)O)CC(=O)O)C3C]

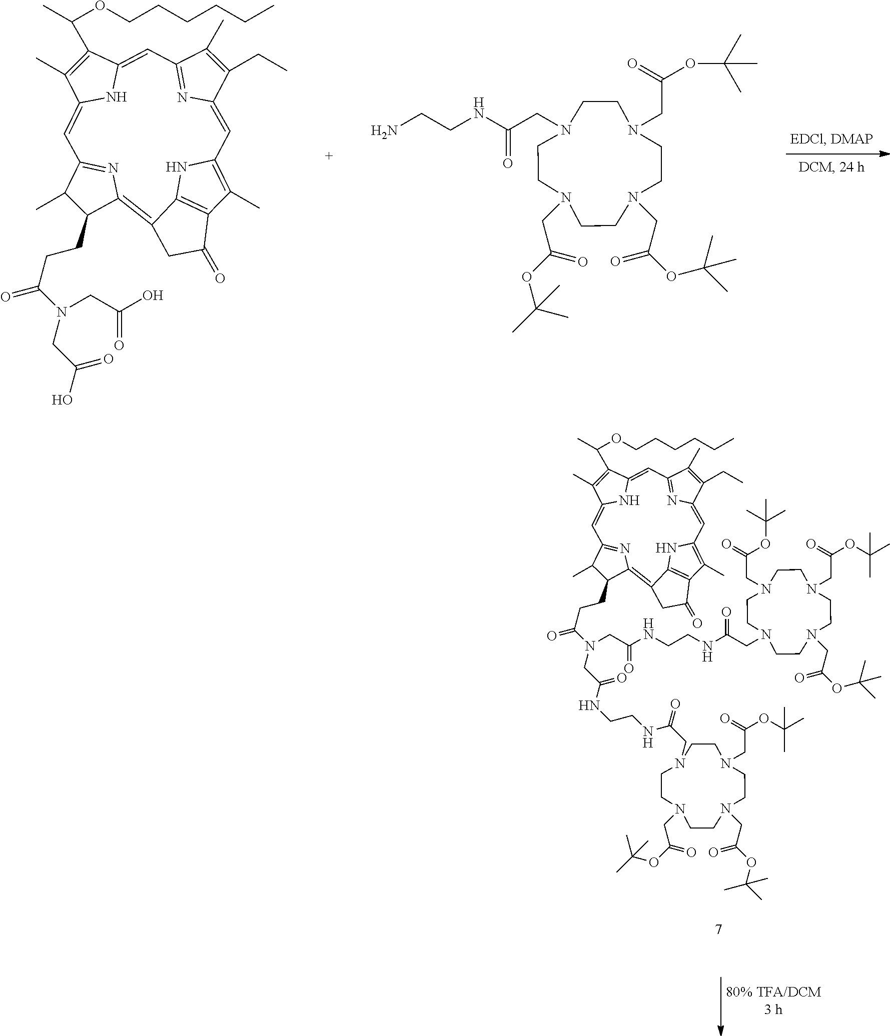 [SMILES: CCCCCCOC(C)c1c(C)c2cc3nc(c4c5[nH]c(cc6nc(cc1[nH]2)C(C)=C6CC)c(C)c5C(=O)C4)[C@@H](CCC(=O)N(CC(=O)O)CC(=O)O)C3C ; CC(C)(C)OC(=O)CN1CCN(CC(=O)NCCN)CCN(CC(=O)OC(C)(C)C)CCN(CC(=O)OC(C)(C)C)CC1 ; CCCCCCOC(C)c1c(C)c2cc3nc(c4c5[nH]c(cc6nc(cc1[nH]2)C(C)=C6CC)c(C)c5C(=O)C4)[C@@H](CCC(=O)N(CC(=O)NCCNC(=O)CN1CCN(CC(=O)OC(C)(C)C)CCN(CC(=O)OC(C)(C)C)CCN(CC(=O)OC(C)(C)C)CC1)CC(=O)NCCNC(=O)CN1CCN(CC(=O)OC(C)(C)C)CCN(CC(=O)OC(C)(C)C)CCN(CC(=O)OC(C)(C)C)CC1)C3C]

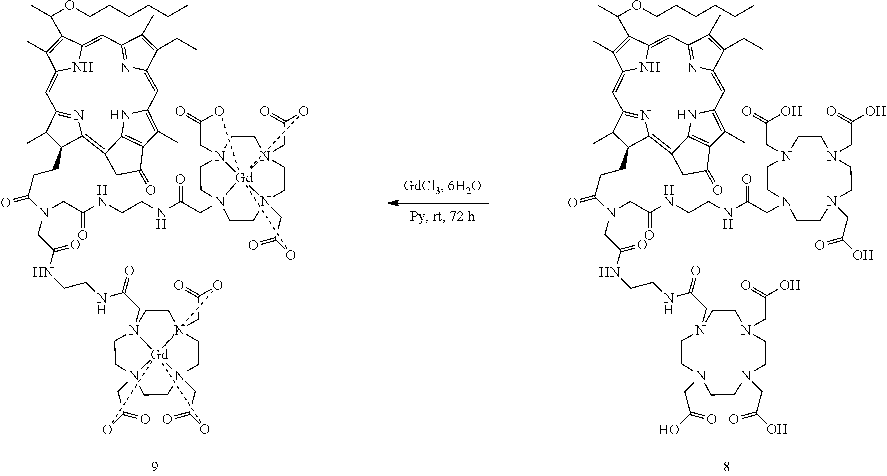

Synthesis of Compound 8: Compound 7 (500 mg, 0.257 mmol) was stirred with 80% trifluoroacetic acid (TFA)/dichloromethane (DCM, 30 mL) at room temperature for 3 h. The resultant mixture was concentrated and the pH was adjusted to 6.5 by adding sat. NaHCO₃ dropwise. The crude reaction mixture was purified by Bio-Gel P-6 (medium) column using DI water as eluent to give product 8 with 90% yield (372.13 mg). MS (ESI) m/z: 802.92×2 (M−H⁺). UV-vis (MeOH, λmax, nm (ε): 659 (0.291), 604 (0.052), 536.9 (0.057), 506 (0.056), 409.1 (0.593), 319 (0.134).

Synthesis of Compound 9: Compound 8 (300 mg, 0.1865 mmol) was dissolved in pyridine (50 mL), and while stirring, GdCl₃.6H2O (277.23 mg, 0.7458 mmol) in 5 mL of DI water added slowly, and the resultant mixture was stirred at room temperature for 72 h. The reaction mixture was concentrated to dryness under high vacuum. The residue was washed with DCM (2×20 mL) and acetone (2×20 mL). The crude reaction mixture was purified by Bio-Gel P-6 (medium) column using DI water twice and 1K 50 mL centrifugal devices for 4 times (each tube 100 mg) as eluent to give product 9 with 85% yield (303.88 mg). MS (ESI) m/z: 640.23×3 (M+H⁺). HRMS (ESI): calcd for C₇₉H₁₁₁Gd₂N₁₇O₁₉ (M+H⁺) 640.2329; found, 640.2343. UV-vis (MeOH, λmax, nm (ε)): 658 (4.4333 10⁴), 601.1 (2.47×10⁵), 535 (2.23×10⁵), 407.9 (2.04×10⁴), 318 (9.79×10⁴).

Synthesis of Compound 10: HPPH (1200 mg, 1.886 mmol), aminotriester (i.e., di-tert-Butyl 4-amino-4-(3-(tert-butoxy)-3-oxopropyl)heptanedioate) (1174.8 mg, 2.829 mmol), EDCI (1-ethyl-3-(3-dimethylaminopropyl)-carbodiimide hydrochloride) (723 mg, 3.772 mmol), and DMAP (4-dimethyl aminopyridine) (460.88 mg, 3.772 mmol) were taken in a dry, round-bottom flask (1000 mL). Dry DCM (300 mL) was added under an argon atmosphere and the reaction mixture was stirred at room temperature for 16 h under an argon atmosphere. After completion of the reaction, which was determined by TLC), the reaction mixture was diluted with DCM (600 mL), washed with brine solution (200 mL), the organic layer was separated, dried over sodium sulfate, and concentrated down to yield crude product, which was purified by silica gel using 1-3% methanol/DCM mixture as eluent to give product 10 with 86% yield (1676.23 mg). ¹H NMR (400 MHz, CDC₃, δ ppm): 9.79 (1H, s, 5-H), 9.48 (1H, s, 10-H), 8.54 (1H, s, 20-H), 5.914/5.910 (1H, q, J=6.8 Hz, 3¹-H), 5.90/5.88 (1H, br s, amide H), 5.299/5.295 (1H, d, J=19.8, 13¹-CHH), 5.12 (1H, d, J=19.8 Hz, 13¹-CHH), 4.52 (1H, q, J=7.1 Hz, 18-H), 4.31 (1H, d, J=7.6 Hz, 17-H), 3.71 (2H, q, J=7.6, 8-CH₂CH₃), 3.63 (2H, m, —OCH₂(CH₂)₄CH₃), 3.61/3.60 (3H, s, 12-CH₃), 3.387/3.385 (3H, s, 2-CH₃), 3.27 (3H, s, 7-CH₃), 2.66 (1H, m, 1H of 17-CH₂CH₂—), 2.34 (1H, m, 1H of 17-CH₂CH₂—), 2.25 (1H, m, 1H of 17-CH₂CH₂—), 2.08-2.16 (9H, m, 3¹-CH₃ & 3 x —CH₂CH₂—COOC(CH₃)₃), 1.85-1.97 (7H, m, 1H of 17-CH₂CH₂— & 3 x —CH₂CH₂—COOC(CH₃)₃), 1.81 (3H, d, J=7.3 Hz, 18-CH₃), 1.74 (2H, m, —OCH₂CH₂(CH₂)₃CH₃), 1.70 (3H, t, J=7.6 Hz, 8-CH₂CH₃), ˜1.38 (2H, m, —O(CH₂)₂CH₂(CH₂)₂CH₃), 1.30 (27H, s, 3x —COOC(CH₃)₃), 1.19-1.26 (4H, m, —O(CH₂)₃(CH₂)₂CH₃), 0.78 (3H, distorted t, J˜7 Hz, —O(CH₂)₅CH₃), 0.45 (1H, br s, core NH), −1.71 (1H, s, core NH); ¹³C (100 MHz, CDCl₃, δ ppm): 196.1, 172.9, 171.7, 171.6, 160.5, 155.1, 150.7, 149.0, 144.9, 141.42/141.37, 139.69/139.66, 137.7, 136.2, 135.6/135.5, 132.32/132.25, 130.5, 128.2, 106.1, 104.0, 97.9, 92.7, 80.6, 72.9/72.8, 69.7, 57.4, 51.8, 50.0, 48.1, 33.81/33.80, 31.7, 30.6, 30.2, 30.1, 29.8, 28.0, 26.1, 24.72/24.71, 23.2/23.1, 22.6, 19.5, 17.4, 14.0, 12.0, 11.3, 11.0. MS (ESI) m/z: 1034.65 (M+H⁺) UV-vis (CH₂Cl₂, λmax, nm (ε)): 660 (0.694), 604 (0.127), 536.9 (0.139), 505.1 (0.132), 407.1 (1.361), 319.0 (0.346).

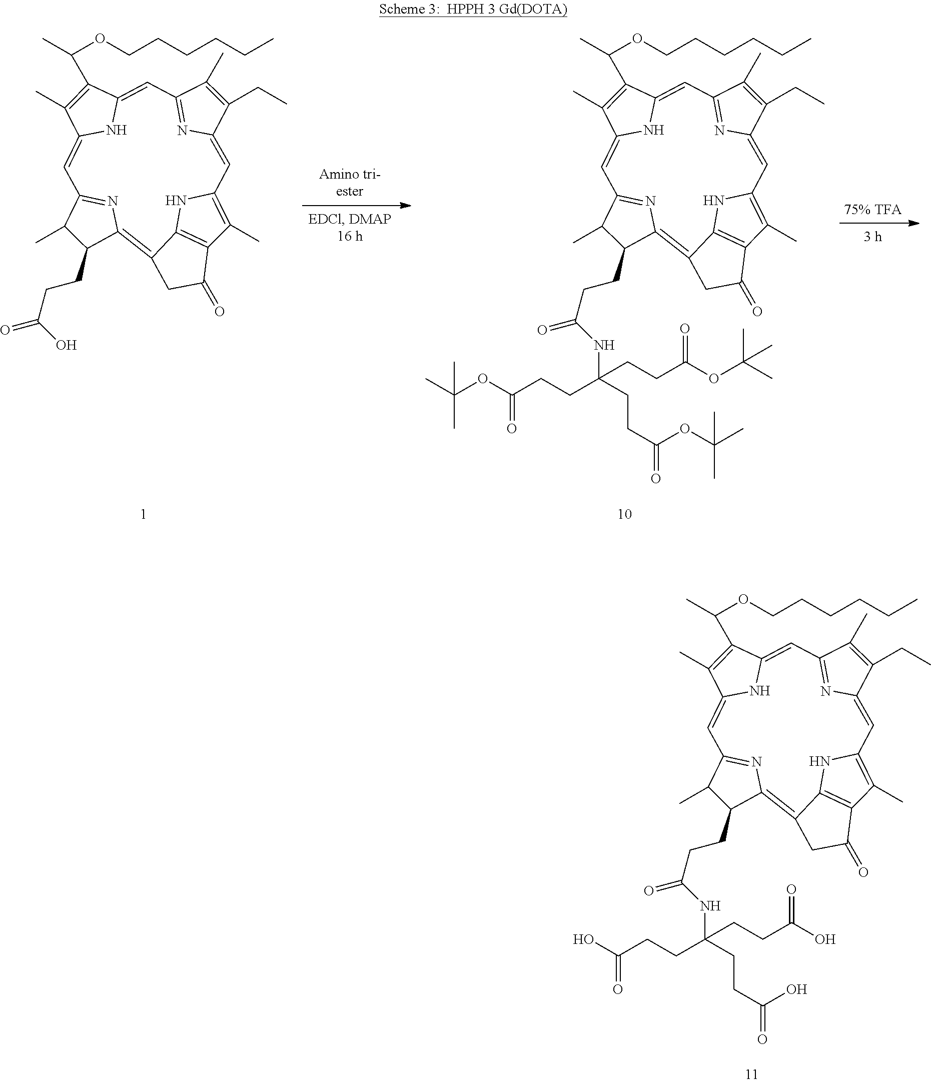 [SMILES: CCCCCCOC(C)c1c(C)c2cc3nc(c4c5[nH]c(cc6nc(cc1[nH]2)C(C)=C6CC)c(C)c5C(=O)C4)[C@@H](CCC(=O)O)C3C ; CCCCCCOC(C)c1c(C)c2cc3nc(c4c5[nH]c(cc6nc(cc1[nH]2)C(C)=C6CC)c(C)c5C(=O)C4)[C@@H](CCC(=O)NC(CCC(=O)OC(C)(C)C)(CCC(=O)OC(C)(C)C)CCC(=O)OC(C)(C)C)C3C ; CCCCCCOC(C)c1c(C)c2cc3nc(c4c5[nH]c(cc6nc(cc1[nH]2)C(C)=C6CC)c(C)c5C(=O)C4)[C@@H](CCC(=O)NC(CCC(=O)O)(CCC(=O)O)CCC(=O)O)C3C]

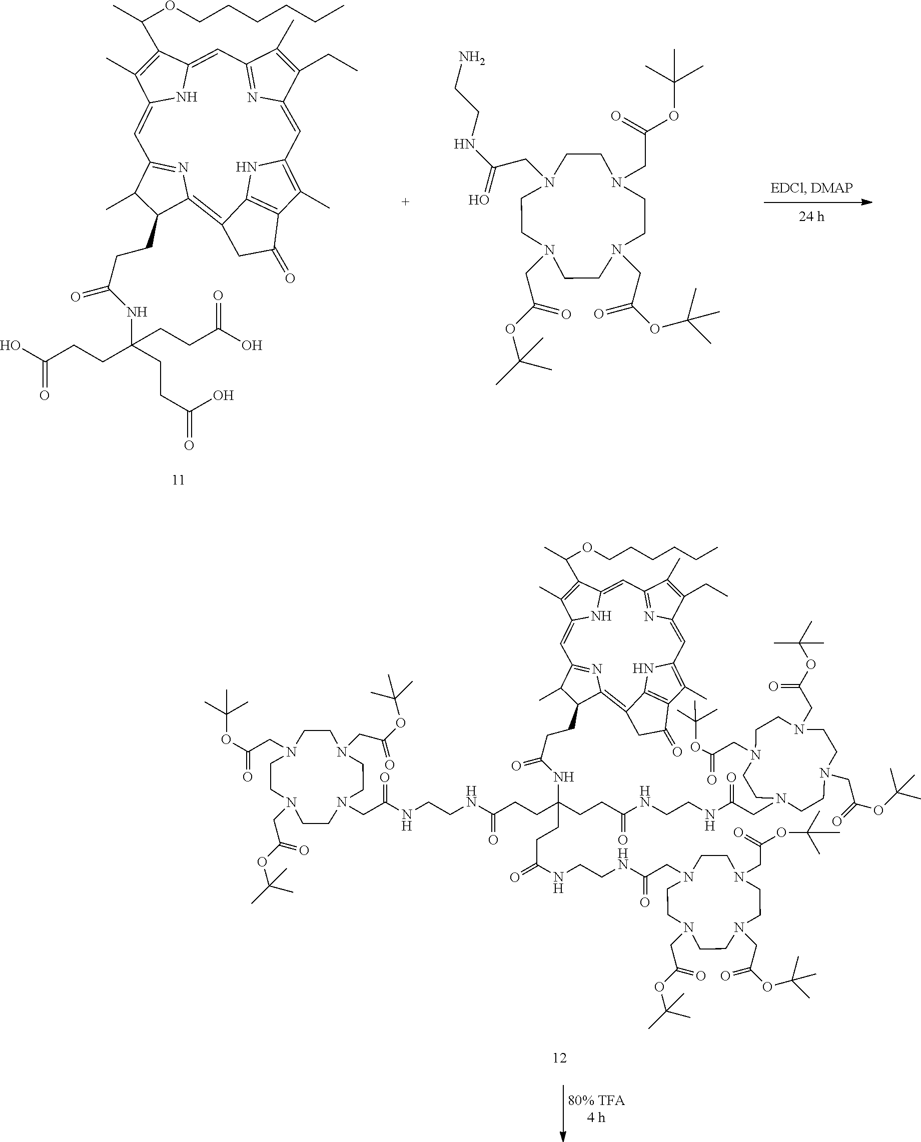

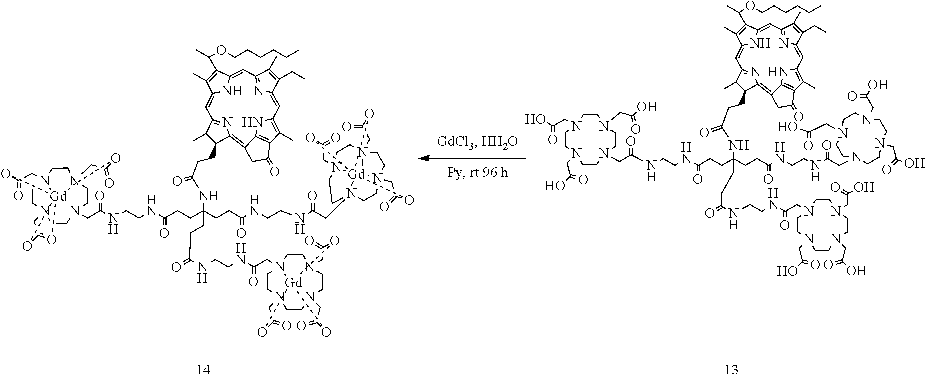

Synthesis of Compound 11: Compound 10 (1600 mg, 1.5469 mmol) was stirred with 75% trifluoroacetic acid (TFA)/dry dichloromethane (DCM, 130 mL) at room temperature for 3 h. The resultant mixture was concentrated and the pH was adjusted to 6.5 by adding sat. NaHCO₃ dropwise and washed with DCM/MeOH (90:10) and DI water, the organic layer was separated dried over sodium sulfate, and concentrated down to yield crude product 11 with 89% yield (1192.29 mg). MS (ESI) m/z: 866.46 (M+H⁺). HRMS (ESI): calcd for C₄₉H₆₃N₅O₉ (M+H⁺) 866.4695; found, 866.4698. UV-vis (MeOH, λₘₐₓ, nm (ε)): 660 (0.754), 604 (0.139), 536.9 (0.149), 506 (0.144), 408 (1.477), 319.0 (0.379).

Synthesis of Compound 12: Dry dichloromethane (400 mL) was added to compound 11 (1100 mg, 1.271 mmol), 2-aminoethyl-DOTA-tris(t-Bu ester) (3.54 gr, 5.081 mmol), EDCI (1217.4 mg, 6.351 mmol), and DMAP (779.3 mg, 6.351 mmol) in a 1000 mL round bottom flask. The reaction mixture was stirred at room temperature for 24 h under an argon atmosphere. After completion of the reaction, which was determine by TLC, the reaction mixture was diluted with DCM (400 mL), washed with brine solution, the organic layer was separated, dried over sodium sulfate, and concentrated down to yield crude product which was purified by Alumina, GRADE III using 4% methanol/DCM mixture as eluent to give product 12 with 78% yield (2631.8 mg). ¹H NMR (400 MHz, CDCl₃, δ ppm): 9.79/9.74 (1H, s, 5-H), 9.47/9.46 (1H, s, 10-H), 8.63/8.62 (1H, s, 20-H), 8.29 (3H, br s, 3 x —NHCH₂CH₂NH—), 8.17 (1H, br s, 17-CH₂CH₂CONH—), 8.06 (3H, br s, 3 x —NHCH₂CH₂NH—), 5.89/5.87 (1H, q, J=7.1 Hz, 3¹-H), 5.34/5.33 (1H, d, J=20.3 Hz, 13¹-CHH), 5.20 (1H, br d, J=20.2 Hz, 13¹-CHH), 4.68 (1H, br q, J˜7 Hz, 18-H), 4.25 (1H, br d, J=10.3 Hz, 17-H), 3.68 (2H, q, J=7.8 Hz, 8-CH₂CH₃), 3.53-3.76 (2H, m, —OCH₂(CH₂)₄CH₃), 3.649/3.646 (3H, s, 12-CH₃), 1.5-3.5 (72H, br m, DOTA ring and exocyclic CH₂ groups, see notes below), 3.38 (3H, s, 2-CH₃), 3.31 (12H, br s, 3 x —NHCH₂CH₂NH—), 3.23 (3H, s, 7-CH₃), 3.12 (1H, m, 1H of 17-CH₂CH₂—), 2.87 (1H, m, 1H of 17-CH₂CH₂—), 2.81 (1H, m, 1H of 17-CH₂CH₂—), 2.41 (6H, br s, 3 x —CH₂NHC═OCH₂CH₂—), 2.14 (6H, br s, 3 x -CH₂NHC═OCH₂CH₂—), 2.11/2.09 (3H, d, J=6.9 Hz, 3¹-CH₃), 2.02 (1H, m, 1H of 17-CH₂CH₂—), 1.78/1.77 (3H, d, J=7.2 Hz, 18-CH₃), 1.74 (2H, m, —OCH₂CH₂(CH₂)₃CH₃), 1.71 (3H, t, J=7.5 Hz, 8-CH₂CH₃), 1.39/1.38/1.36 (81H, 3 x s, 9 x —COOC(CH₃)₃), ˜1.38 (2H, m, —O(CH₂)₂CH₂(CH₂)₂CH₃), 1.18-1.29 (4H, m, —O(CH₂)₃(CH₂)₂CH₃), 0.83/0.76 (3H, distorted t, J˜7 Hz, —O(CH₂)₅CH₃), 0.33/0.31 (1H, s, core NH), −1.86/−1.89 (1H, s, core NH); ¹³C NMR: (100 MHz, CDCl₃, δ ppm): 196.1, 174.3, 173.6, 173.01/172.98, 172.3, 172.2, 171.3, 162.72/162.69, 154.9, 150.53/150.51, 148.83/148.82, 144.7, 141.2/141.1, 139.7/139.5, 137.43/137.41, 136.3/136.2, 135.6/135.5, 132.9/132.7, 130.61/130.58, 127.84/127.77, 106.5/106.4, 103.4, 97.7/97.6, 93.9, 81.75, 81.68, 72.83/72.79, 69.8, 58.3/58.2, 56.0, 55.5, 55.4, 46-54 (see notes below), 52.5, 50.22/50.21, 48.3, 39.3, 38.7, 35.2, 33.1, 33.1, 31.80/31.70, 31.74, 30.3/30.2, 28.0, 27.9, 26.2/26.1, 25.1/24.6, 23.44/23.42, 22.6/22.5, 19.5, 17.7, 14.0/13.9, 12.0, 11.31/11.30, 11.13/11.09. MS (ESI) m/z: 664.94×4 (M+H⁺). HRMS (ESI): calcd for C₁₃₉H₂₃₁N₂₃O₂₇ (M+H⁺) 664.9445×4; found, 664.9431×4. UV-vis (CH₂Cl₂, λₘₐₓ, nm (ε)): 669 (4.45×10⁴), 659 (0.240), 604 (0.043), 536 (0.047), 506 (0.046), 408 (0.483), 319 (0.108).

Synthesis of Compound 13: Compound 12 (2500 mg, 0.9411 mmol) was stirred with 80% trifluoroacetic acid (TFA)/dichloromethane (DCM, 240 mL) at room temperature for 4 h. The resultant mixture was concentrated and the pH was adjusted to 6.5 by adding sat. NaHCO₃ dropwise. The crude reaction mixture was purified by Bio-Gel P-6 (medium) column using DI water as eluent to give product 13 with 89% yield (1802.06 mg). MS (ESI) m/z: 538.80×4 (M+H⁺). HRMS (ESI): calcd for C₁₀₃H₁₅₉N₂₃O₂₇ (M+H⁺) 538.8019×4; found, 538.8036×4. UV-vis (CH₂Cl₂, λₘₐₓ, nm (ε)): 660 (0.654), 603 (0.127), 536.9 (0.129), 505.1 (0.124), 408 (1.285), 319 (0.339).

Synthesis of Compound 14: Compound 13 (1600 mg, 0.4183 mmol) was dissolved in pyridine (150 mL), and while stirring, GdCl₃.6H₂O (1658.50 mg, 4.462 mmol) in 15 mL of DI water added slowly, and the resultant mixture was stirred at room temperature for 96 h. The reaction mixture was concentrated to dryness under high vacuum. The residue was washed with DCM (2×60 mL) and acetone (2×60 mL). The crude reaction mixture was purified by Bio-Gel P-6 (medium) column using DI water twice and 1K 50 mL centrifugal devices for 4 times (each tube 100 mg) as eluent to give product 14 with 86% yield (1672 mg). MS (ESI) m/z: 872.6×3 (M⁺). UV-vis (MeOH, λmax, nm (ε)): 659 (4.54×10⁴), 603 (2.58 ×10⁵), 536 (2.41×10⁵), 505 (2.27×10⁵), 409 (2.12×10⁴).

Spin filtration. To the top portion of each of Macrosep® Advance Centifugal Devices (1000 MWCO) was added 100 mg of crude HPPH-DOTA-Dp and HPPH-DOTA-Gd and 22 mL of DI water. Tubes were placed in a centrifuge, spun at 4150 rpm for 40 minutes. When spinning stopped, the filtrate was removed from the lower portion of the device.

A new portion of 20 mL DI water was added to the top and the centrifuging process repeated. The spin filtration process was done a total of four times. After the fourth centrifuging, the retentates from the all tubes were combined into one Macrosep® Advance tube and centrifuged. The combined retentive was dissolved with minimum volume of DI water.

### EXAMPLE 2

This example provides a description of characterization of compounds of the present disclosure, methods of making compounds of the present disclosure, and uses of compounds of the present disclosure.

Determination of free gadolinium in solutions of HPPH-DOTAₙ-Gdₙ. To determine if free Gd³⁺ was present in a batch of HPPH-DOTAₙ-Gdₙ (n=1, 2, or 3) the Xylenol Orange assay was used. In 50 mM acetate buffer at pH 5.8, Xylenol Orange has a main absorption peak maxima at approximately 433 nm and a weaker absorption at approximately 570 nm. As the Gd⁺ concentration increases the 433 nm band decreases in intensity (and shifts in the region to somewhere around 432-438 nm); simultaneously the peak at 570 nm increases in intensity (with a slight shift in position of the peak maxima to somewhere around 565 to 573 nm). HPPH has absorption in these regions so the free [Gd³⁺] in a solution of HPPH-DOTAₙ-Gdₙ was measured indirectly by spin-filtering a solution of HPPH-DOTAₙ-Gdₙ using a centrifugal spin-filter device and using the clear filtrate obtained as the sample in the Xylenol Orange assay.

As described in detail herein, a Xylenol Orange solution in 50 mM acetate buffer at pH 5.8 was prepared. Standard solutions of Gd³⁺ with concentrations of 0, 10, 20, 30, 40, 50 μM were prepared.

When gadolinium standard solutions are mixed with Xylenol Orange, the spectra from 300-1000 nM of the complex formed were obtained at each concentration of Gd³⁺. The free [Gd³⁺] is proportional to the ratio of the peak maxima of the longer wave length band (maxima between 565 to 573 nm) to the shorter wavelength band (maxima between 432 nm to 438 nm). From this a standard curve was plotted and best-fit linear equation calculated.

The spin-filter filtrates of samples tested were mixed with Xylenol Orange and the UV-vis spectra were obtained. The free [Gd³⁺] of a sample was calculated off the standard curve using the sample's (Abs₅₆₇ₙₘ/Abs₄₃₃ₙₘ) ratio.

### Xylenol Orange Test Procedure

### I. Preparation of Xylenol Orange Test Solution

a) 50 mM acetate buffer was prepared by adding 1.44 mL acetic acid in 400 mL distilled deionized (dd) H₂O, and the pH was adjusted to 5.8 with 1 M NaOH. The volume was brought to 500 mL with dd H₂O; and

b) 6 mg Xylenol Orange was added to 100 mL of 50 mM acetate buffer.

### II. Preparation of Gadolinium Standard Solutions (Using Gadolinium Trichloride Hexahydrate

a) A 5 mM stock was prepared by adding 46.5 mg GdCl₃.6H₂O to 25 mL dd H₂O;

b) A 1 mM stock was prepared by diluting 10 mL of 5 mM stock 50 mL dd H₂O; and

c) The following solutions were prepared: 10, 20, 30, 40, and 50 uM Gd using 1 mM stock and dd water.

TABLE 1
Gd (III) solution set recipe dilutions:

| Volume 1 | Volume | Final |
|---|---|---|
| mM Stock | water | concentration |
| 2.5 mL | 47.5 mL | 50 μM |
| 2 mL | 48 mL | 40 μM |
| 1.5 mL | 48.5 mL | 30 μM |
| 1 mL | 49 mL | 120 μM |
| 0.5 mL | 49.5 mL | 10 μM |

### III. Preparation of HPPH-DOTAₙ-Gdₙ Solutions for Gd Content

a) HPPH-3DOTA-3Gd (solid batch #RPCI973061716) fresh solution, 5.8 mM; 96 mg was dissolved in 6 mL DPBS, filtered through a 0.2 μm syringe filter. 6 ml spinfiltered through Macrosep® Centrifugal filter device, 1 kDa MWCO, Part # MAP001C37. Volume of filtrate was 2.8 mL.

b) HPPH-3DOTA-3Gd (solid batch # RPCI973061716) 2.8 mL of solution from rabbit test, conc=6.5 mM (235 mg dissolved in 14.7 mL DPBS, filtered through 0.2 μm syringe filter. Spin-filtered through Macrosep® Centrifugal filter device, 1 kDa MWCO, Part # MAP001C37. The volume of filtrate was 1.5 mL.

c) HPPH-2DOTA-2Gd (solid batch # RPCI991100115) 7.8 mM, 60 mg dissolved in 4 mL DPBS, filtered through 0.2 μm syringe filter. Spin-filtered through Macrosep® Centrifugal filter device, 1 kDa MWCO, Part # MAP001C37. The volume of filtrate was 2.5 mL.

d) HPPH-1DOTA-1Gd (solid batch # RPCI611081815) 4.1 mM, 40 mg dissolved in 8 ml DPBS, filtered through 0.2 μm syringe filter. Spin-filtered through Macrosep® Centrifugal filter device, 1 kDa MWCO, Part # MAP001C37. The volume of filtrate was 5 mL.

The volume of filtrate was recorded because not all liquid may pass through the filter. Therefore this may be taken into account when calculating the amount of free Gadolinium once the concentration of free Gadolinium in the filtrate is determined.

### IV. Xylenol Orange Assay to Measure Free Gadolinium

1. In individual 2 mL tubes, 1.5 mL of xylenol orange solution was mixed with 0.5 mL of each standard Gd solution.
2. For 0 μM Gd, in another 2 mL tube, 1.5 mL xylenol orange solution was mixed with 0.5 mL dd H₂O.
3. Used acetate buffer as the baseline, using the UV-Vis spectrophotometer, collected the spectrum of the above solutions as is from 300 to 1000 nm. Tabulate the peak maxima in the 432 to 438 nm (region A) and 565 to 573 nm (region B).
4. For standard curve, calculated the ratio of the absorbance peak maxima (Abs region B/Abs region A). Ratio (Y) vs concentration (X) was plotted. The best-fit linear equation was obtained.
5. Obtained the spectrum of the filtrate from the spin-filtration (1 kDa MWCO) of the HPPH-DOTAₙ-Gdₙ samples using the same procedure. (1.5 mL xylenol orange solution mixed with 0.5 mL filtrate). The free [Gd³⁺] of a sample was calculated off the standard curve using the sample's (Abs₅₆₇ₙₘ/Abs₄₃₃ₙₘ) ratio.

### Results and Discussion

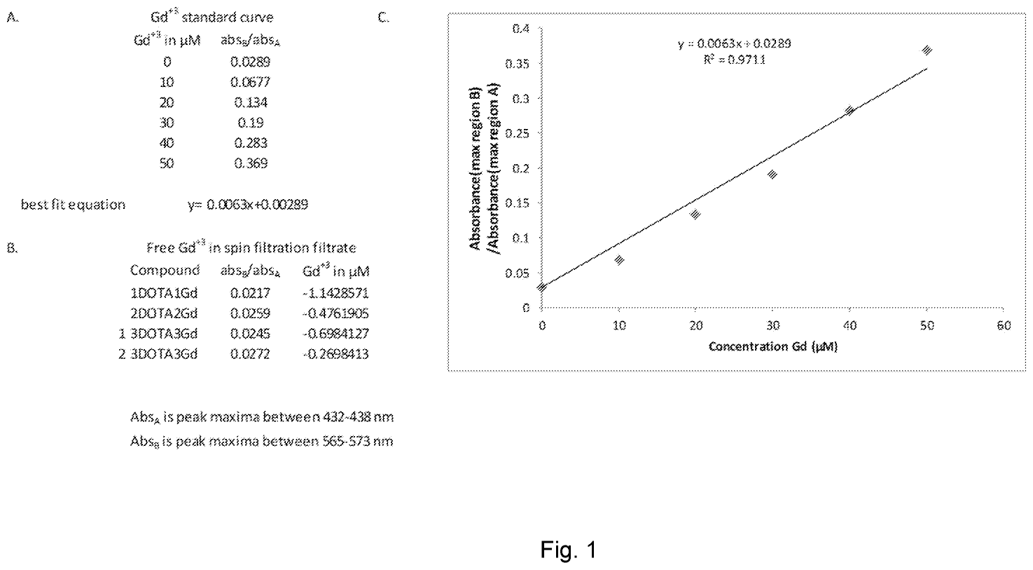

FIG. 1 shows the determination of Gd³⁺ complexed by Xylenol Orange, both the table of data and the best-fit plot. It also shows the calculated free Gd concentrations of HPPH-DOTAₙ-Gdₙ (n=1, 2, and 3). For all the HPPH-DOTAₙ-Gdₙ compounds the ratio of the absorbances was at or below the observed absorbance ratio for [Gd³⁺]=0 μM; i.e. at the level of detection (of the order of 1 ppm). There was no free Gadolinium ion in solutions of these compounds. The level of detection of free Gadolinium is described herein.

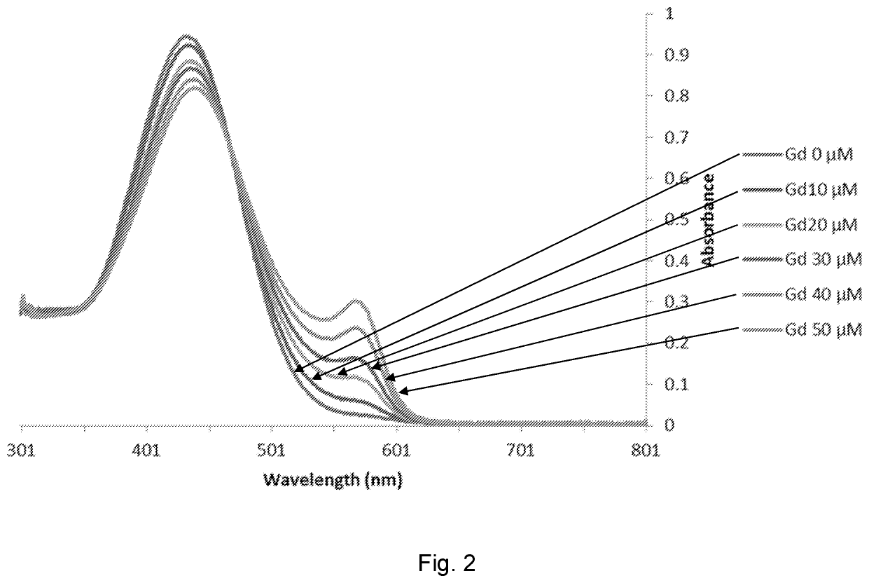

FIG. 2 shows the overlay of the UV-vis absorption spectra of the standard Gadolinium solutions mixed with Xylenol Orange solution, with [Gd³⁺]=0, 10, 20, 30, 40 and 50 μM.

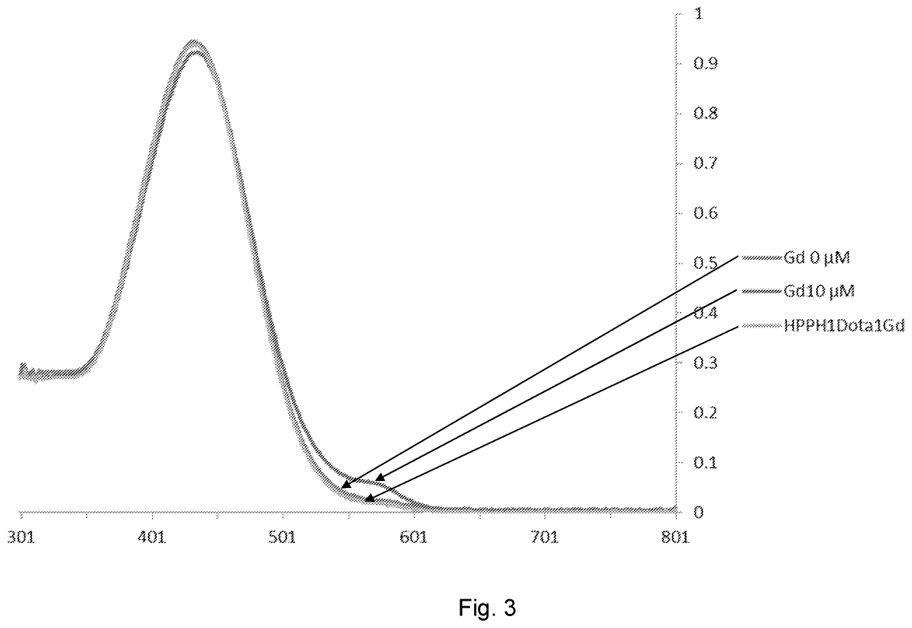

FIG. 3 shows the overlay of the spectrum of the HPPH-DOTA1-Gd1 spin-filtration's filtrate complexed with Xylenol Orange as well the Xylenol Orange test solutions with standard [Gd³⁺]=0 μM and 10 μM.

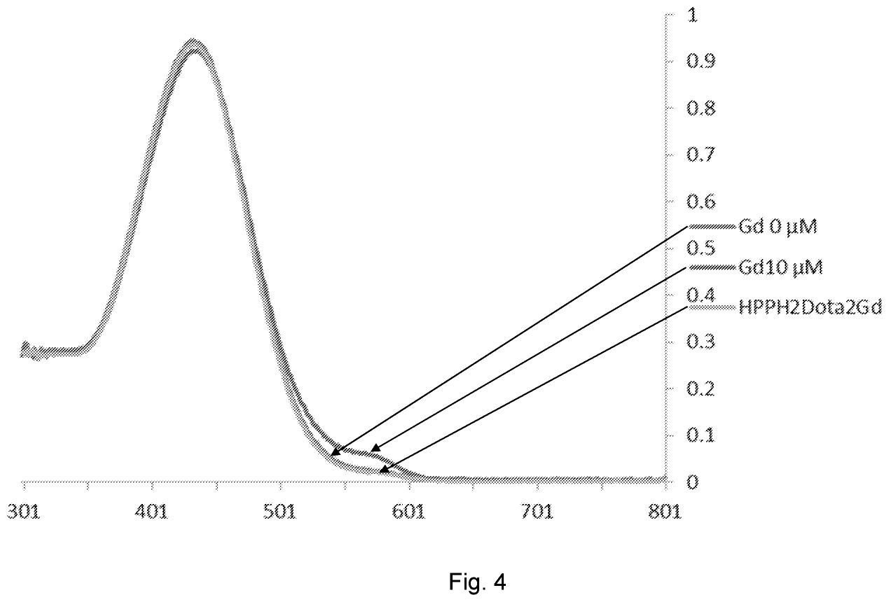

FIG. 4 shows the overlay of the spectrum of the HPPH-DOTA2-Gd2 spin-filtration's filtrate complexed with Xylenol Orange as well the Xylenol Orange test solutions with standard [Gd³⁺]=0 μM and 10 μM.

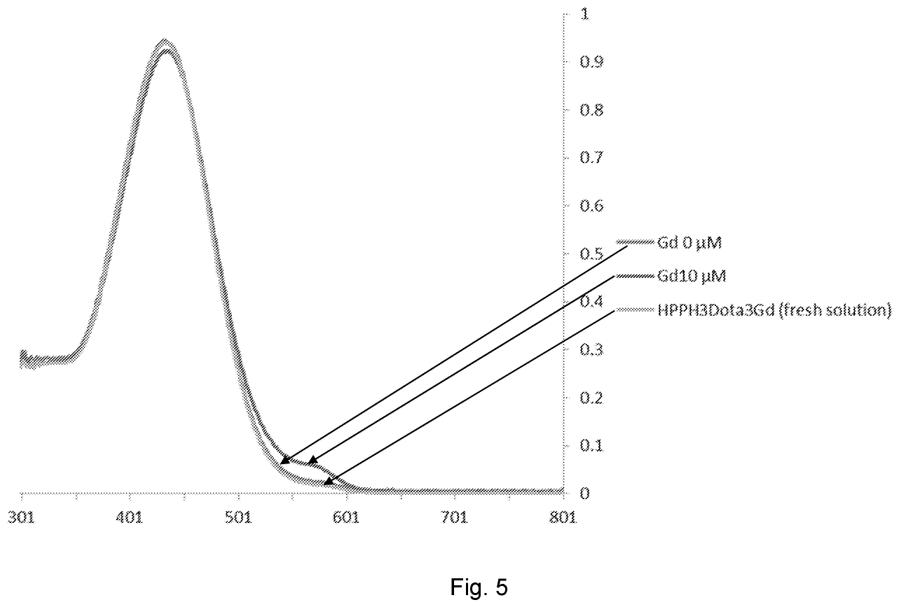

FIG. 5 shows the overlay of the spectrum of the HPPH-DOTA3-Gd3 (solution prepared the same day as the Xylenol Orange assay) spin-filtration's filtrate complexed with Xylenol Orange of Xylenol Orange assay) as well the Xylenol Orange test solutions with standard [Gd³⁺]=0 μM and 10 μM.

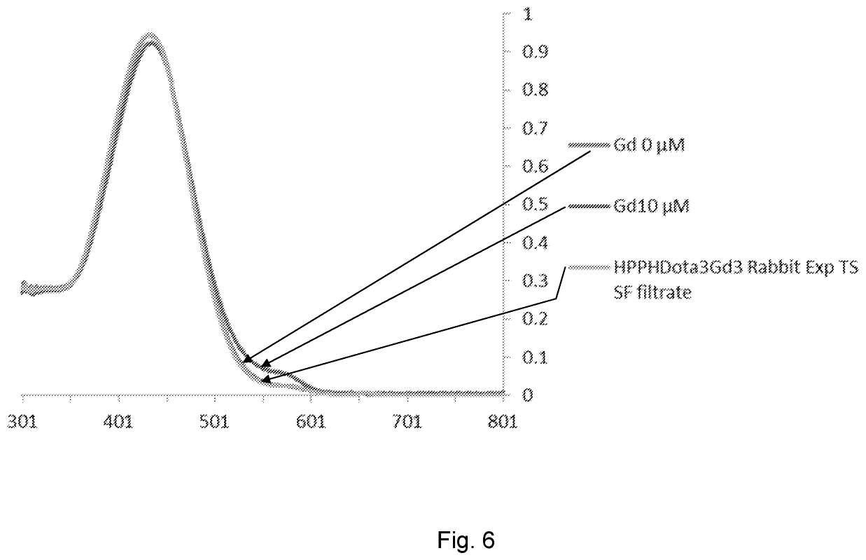

FIG. 6 shows the overlay of the spectrum of the HPPH-DOTA3-Gd3 (solution from Rabbit MRI experiment) spin-filtration's filtrate complexed with Xylenol Orange as well the Xylenol Orange test solutions with standard [Gd³⁺]=0 μM and 10 μM.

Detection Level of Free Gd³⁺ in HPPH-DOTAₙ-Gdₙ solutions using the Xylenol Orange Assay. Using the 10 μM standard as an example, 10 μM is equivalent to 0.01 μmol/mL. The atomic weight of Gadolinium is 157 g/mol. 0.01 μmol/mL×157 μg/μmol=1.57 μg/mL. 10 μM Gadolinium corresponds to 1.57 μg/mL and 1 μM corresponds to 0.157 μg/mL.

In the Xylenol Orange test, a volume of 0.5 ml of standard solution is used for each measurement. At 10 μM this corresponds to approximately 0.8 μg Gd in the solution measured. The UV-vis spectral response amount to this can be seen in FIGS. 1, 3, 4, 5, and 6. The 1 μM level of Gadolinium would correspond to 0.08 μg free Gd. For example, if the free Gadolinium concentration as determined from the standard curve was 1 μM and the total compound sample weight was 96 mg, then 0.08 μg/96000 μg is 0.8 ppm free Gd.

### EXAMPLE 3

This example provides a description of characterization of compounds of the present disclosure, methods of making compounds of the present disclosure, and uses of compounds of the present disclosure.

Magnetic resonance imaging (MRI) is a powerful clinical imaging modality that offers superior soft-tissue contrast without the presence of ionizing radiation. Signal arises in MRI through the difference in spin/energy states of magnetically-active nuclei within a magnetic field (B₀), most commonly hydrogen nuclei due to its intrinsic high sensitivity and abundance in biological substances, e.g. water and lipids. An excess of nuclei in the lower energy state creates a bulk magnetization within tissues, known as the net magnetization vector (NMV). Angular momentum of the nuclear magnetic dipoles creates a precession of the nuclear spins around the magnetic field, with the processional frequency (Larmor frequency) linearly dependent on the strength of the magnetic field. Application of non-resonance radiofrequency waves imparts energy into the spin system, tipping the NMV away from the B₀. Precession of the NMV induces alternating current into a MRI coil with a frequency of the Larmor frequency that is later digitized for reconstruction by Fourier transform. Application of magnetic field gradients in any combination of the three orthogonal directions allow for spatial encoding of the nuclei's frequency and phase of precession, resulting in a multidimensional image comprised of a number of user-prescribed voxels.

The intensity of signal arising from each encoded voxel is largely dependent upon five or six factors: (a) hydrogen density, N_{H} (b) T1 relaxation time of the nuclei imaged (c) T2 relaxation time of the nuclei imaged (d) operator defined “echo time” or “TE” and I operator-defined “repetition time” or “TR” and (f) and nutation or flip angle “FA” (Θ), which is the number of radians the NMZ is tipped away from the static magnetic field B₀ (used primarily in gradient-recalled echo imaging).

T1 relaxation is the process in which the excited nuclei release energy to the surrounding lattice. As energy is released the NMV re-aligns with the magnetic field B₀, as described by the Bloch equation Mz=M₀(1−e^{-t}/T1), where Mz=proportion of NMV aligned in the direction of the magnetic field, M₀ is the strength of the NMV, t=time after excitation of the nuclei and T1 is defined as the time required for 63% of M₀ to align with the magnetic field in the z-direction. T2 relaxation is the process in which the nuclear spins interact with each other and localized differences in the magnetic field, which causes variation in the precessional frequencies and loss of phase coherence. This process is described mathematically by the other Bloch equation, Mxy=M₀(e^{-t}/T2), where M_{xy} is the proportion of M₀ in the transverse (x-y) plane, t=time after excitation and T2 is the time required for Mxy to decay to 37% of original value after excitation. In general, shorter T1 times result in greater signal, while shorter T2 times result in less signal from a specific tissue.

Acquisition parameters such as echo and repetition times can be exploited to weight contrast in the image based upon differences in either proton density or T1/T2 times. The echo time is the time after initial excitation that the encoded signal is sampled and repetition time is the time between successive excitations required for spatial encoding. Signal arising from tissue can be approximated by Equation 1 for “spin-echo” MRI scans or Equation 2 for “spoiled gradient-recalled echo” MRI acquisitions (SGPR).

$$\begin{matrix}SI α {N}_{H} * ( 1 - {e}^{- TR / T 1} ) * ( {e}^{- TE / T 2} ) & Eq . 1 \\ SI α {N}_{H} * \frac{sin θ * ( 1 - {e}^{- TR / T 1} ) * ( {e}^{- TE / T 2} )}{1 - cos θ * ( {e}^{- TR / T 1} )} & Eq . 2\end{matrix}$$

As such, the operator can acquire a “T1-weighted” (T1W) scan by utilizing a short TE to reduce time T2 relaxation is allowed to occur, and use a short TR to accentuate differences in T1 relaxation rates. For T1W-SPGR scans, as large flip angle (>30°) is used as well.

A tissue's T1 and T2 times are dependent upon a number of factors, such as the degree of mobility of water molecules, concentrations of proteins, concentration of lipid hydrocarbons and concentration of paramagnetic metals. To modify a tissue's T1 and T2 times, a MRI “contrast agent” is administered, generally intravenously, containing a gadolinium (III) ion within a chelation molecule to reduce toxicity. Gadolinium (III) has 7 unpaired electron in inner orbital shells, imparting a high degree of paramagnetism which induces faster T1 and T2 relaxation of nearby hydrogen nuclei. While gadolinium-based contrast agents (GBCA's) accelerate both T1 and T2 relaxation processes, they are often considered “T1 agents” as there is an overall positive signal change in tissues with GCBA's due to (a) use of T1-weighted imaging as opposed T2-weighted and (b) T1 relaxation is often a magnitude slower than T2 relaxation, and therefore the percent change in T1 times are much greater than the percent change in T2 time.

Clinically-approved MRI contrast agents are non-specific and their distribution is controlled primarily by degree of tissue vascularity. As a result, MRI agents seldom strongly enhance the signal of tumors due to poor intratumoral perfusion, with the exception of glioma brain tumors, where enhancement is due to the loss of the blood-brain barrier. As a result, GCBA's that selectively accumulate in tumors would be a significant enhancement to clinical screening for non-localized metastases through the use of T1-weighted MR imaging.

### Imaging Methods

Characterization of MR imaging efficacy for compounds 4, 9 & 14. MR imaging was performed on a 4.7 Tesla preclinical MRI using the ParaVision 3.0.2 acquisition platform (Bruker Biospin, Billerica Mass.). In vitro relaxivities were acquired using the Bruker G060 gradient insert and a quadrature radiofrequency coil (35 mm ID, m2m Imaging, Cleveland Ohio). In vivo imaging in rat models utilized larger Accustar® shield gradients (150 mm ID) and a quadrature radiofrequency coil (72 mm ID, Bruker Biospin).

T1 and T2 relaxivities for compounds 4, 9 & 14 were acquired at 37C. Samples were diluted in PBS with concentrations ranging from 50 to 200 μM and transferred to NMR tubes for imaging. A tube containing 1× PBS was included as a “0 μM control”. T1 relaxation rates of serial dilutions were measured using an inversion-recovery TrueFISP acquisition with the following parameters: TE/TR=1.5/3.0 ms, flip angle=30°, inv. Repetition time=10 s, segments=8, frames=100. T2 relaxation rates were measured using a multi-echo, Carr-Purcell-Meiboom-Gill (CPMG) spin-echo sequence with a fixed TR of 3000 ms and TE times ranging from 20-1200 ms in 20 ms increments (60 echoes). The relaxation rate of each sample calculated using non-linear regression analysis routines developed in MATLAB (MathWorks, Natick, Mass.) and relaxivities were then calculated by linear regression (concentration vs. relaxation rate).

In vivo characterization of compound 14. Fischer 344/NHsd female rats (Envigo) were injected with Ward rat tumor cells (2-3×10⁶) intraperitoneally (IP) and screened by MRI weekly for 1-3 weeks until intraperitoneal tumor growths were detected. A series of T1-weighed spoiled-gradient scans were acquired to characterized tumor and normal tissue enhancement (Table 2). To normalize signal intensities between imaging session, four phantom tubes containing 1% agarose with increasing concentrations of copper sulfate (0 to 3 mM) were imaged alongside the animal. Imaging was performed prior to administration of contrast agent and subsequently repeated immediately, 4 hours and 24 hours after injection of the agent dose of 10 μmol/kg, intravenously via tail vein. To estimate agent concentration within the tumor, T1 relaxometry was performed using a variable TR spin echo scan with RARE-encoding with the following parameters: effective TE=25 ms, RARE factor=4, FOV=6×6 cm, slice thickness=1.5 mm, variable TRs=350 to 6000 ms.

Following MR imaging at 24 hours, animals were euthanized, and fluorescence imaging was performed during necropsy using an IVIS Spectrum imager (640/680 ex/em, Perkin-Elmer).

TABLE 2

| | | | | | Slab/ | | Field | |
|---|---|---|---|---|---|---|---|---|
| | Fat | TE/ | | | Slice | | of | |
| Scan | Satura- | TR | Flip | Matrix | Thick- | | View | |
| Name | tion | (ms) | Angle | Size | ness | | (cm) | NEX |
| 3D-SPGR | No | 3/15 | 60° | 192 × 96 × | 6 | cm | 10.0 × | 2 |
| | | | | 96 | | | 6.0 | |
| 3D-SPGR- | Yes | 3/25 | 60° | 192 × 96 × | 6 | cm | 10.0 × | 2 |
| FATSAT | | | | 96 | | | 6.0 | |
| 2D-SPGR | No | 2.2/ | 90° | 128 × 128 | 2 | mm | 6.0 × | 4 |
| | | 150 | | | | | 6.0 | |

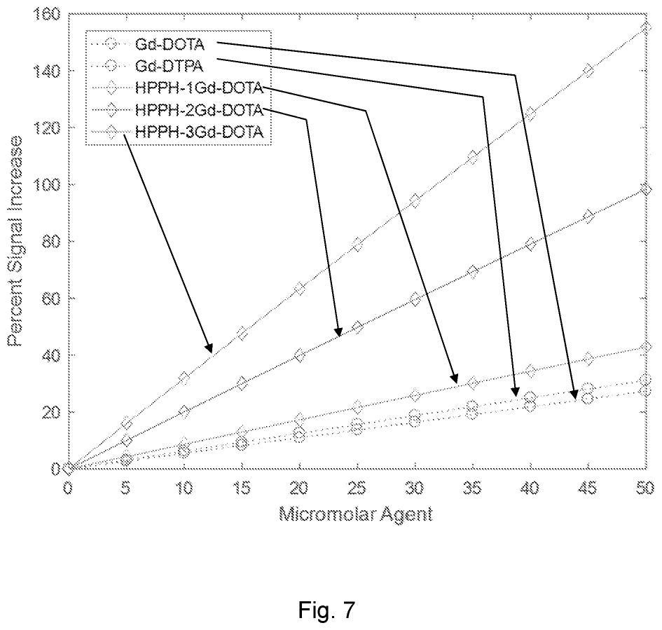

Results. T1 and T2 relaxivities of compounds 4, 9 & 14 are listed in Table 3, as well as the published values of clinically relevant GCBA's at 4.7 T, 37 C. T1 relativities of the compounds increased nearly linearly with additional Gd-DOTA conjugates and all exceeded the published relaxivities of clinically-available contrast agents Gd-DTPA (Magnevist®) and Gd-DOTA (Dotarem®). Moreover, with increasing Gd-DOTA groups conjugated to HPPH, the ratio of T2 to T1 relaxivity (r2/r1) decreases, likely due to the increased hydrophilicity of the compounds with increasing DOTA groups, reducing potential aggregation of the porphyrin rings. For T1-weighted acquisitions, the decrease in T2 time with the administration of the agent will have minimal effect on the signal intensity when compared to the effect of decreased T1 times. In silica modelling of the signal intensity equation for T1-weighted, SPGR scans (Eq. 2) clearly show superior enhancement by compounds 1-3 over clinical agents at equimolar concentrations (FIG. 7).

TABLE 3

| | rl at 4.7T | r2 at 4.7T | |
|---|---|---|---|
| Compound | (mM · s)⁻¹ | (mM · s)⁻¹ | r2/r1 |
| HPPH-(1)-Gd-DOTA | 4.44 | 16.41 | 3.69 |
| HPPH-(2)-Gd-DOTA | 10.65 | 36.02 | 3.38 |
| HPPH-(3)-Gd-DOTA | 16.39 | 33.77 | 2.06 |
| Gd-DOTA † | 2.8 | 3.7 | 1.32 |
| Gd-DTPA † | 3.2 | 4.0 | 1.25 |

† As reported by Rohrer et al., Invest Radiol. 2005 Nov; 40(11):715-24.

In vivo imaging demonstrated strong enhancement in the liver and kidney, as expected due to their high degree of vascularity, which decayed over time due to renal clearance from the blood stream (FIG. 8). Tumor signal enhancement was highest, however, at four hours and compared favorably to muscle. At its peak enhancement at 4 hrs, tumor signal intensity increased by 73% (T1W-SGPR with fat saturation), while muscle showed only minor enhancement of 10% (Table 4). Furthermore, T1 relaxometry reported an increase in the tumor T1 rate of 0.34 (s)⁻¹, corresponding to an intratumoral concentration of 21 μM, while T1 relaxation rates in muscle were virtual unchanged after injection.

TABLE 4

| | Tumor (n = 3) | Muscle (n = 3 |
|---|---|---|
| Timepoint | AU. Signal (% increase) | A.U. Signal (% increase) |
| Baseline | 258 | 314 |
| 15 min | 392 (52%) | 336 (7%) |
| 4 hrs | 445 (73%) | 344 (10%) |
| 24 hrs | 383 (49%) | 341 (9%) |

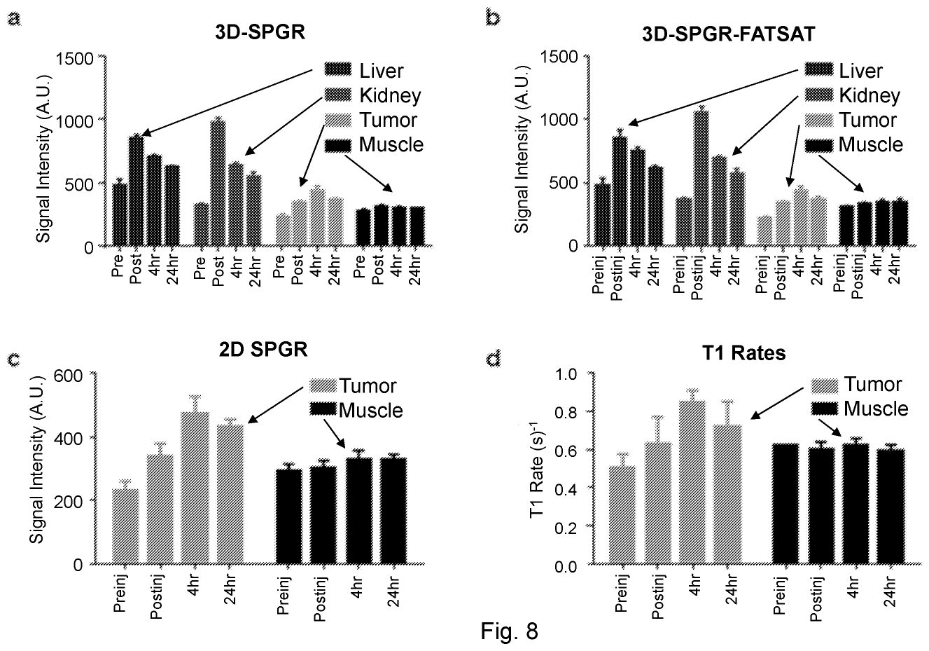

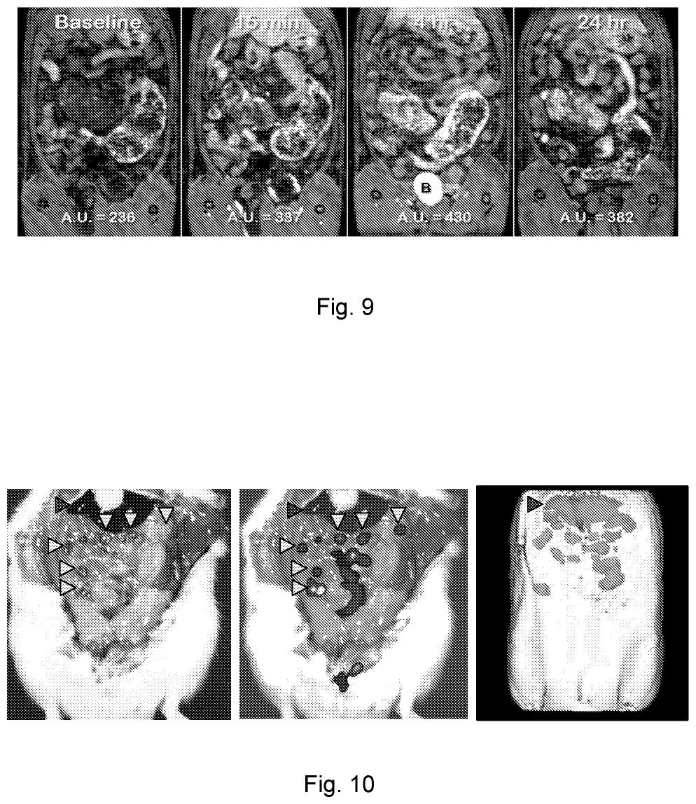

Representative images from fat-suppressed, T1W SPGR scans shows visible enhancement in mesentery growths, many of which are non-distinct prior to injection (FIG. 9).

Three-dimensional volumetric rendering of growths detected by MRI showed strong correlation with post-mortem fluorescence imaging (FIG. 10).

### EXAMPLE 4

This example provides a description of the use compounds of the present disclosure.

Tumor-Avid 3Gd (DOTA)-HPPH Defines Metastases as Small as 1.5 mm in Adult Rabbits at 1.5 Tesla. A POTENTIAL REPLACEMENT FOR F-18-FDG-PET/CT AND F-18-FDG-PET/MRI.

3(Gd)-DOTA-HPPH overcomes the disadvantages of 3(Gd)-DTPA-HPPH: It is water-soluble, can be reconstituted immediately before IV administration, and remains photoactive.

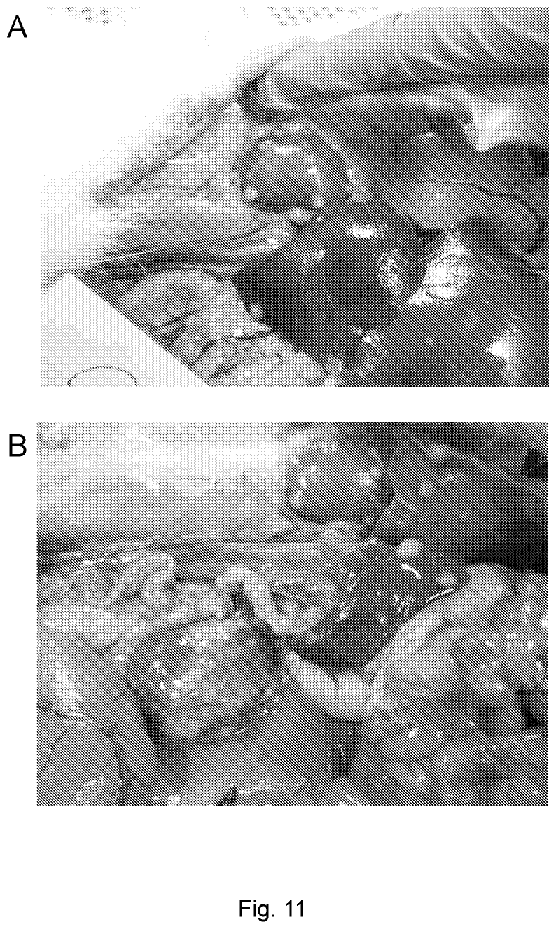

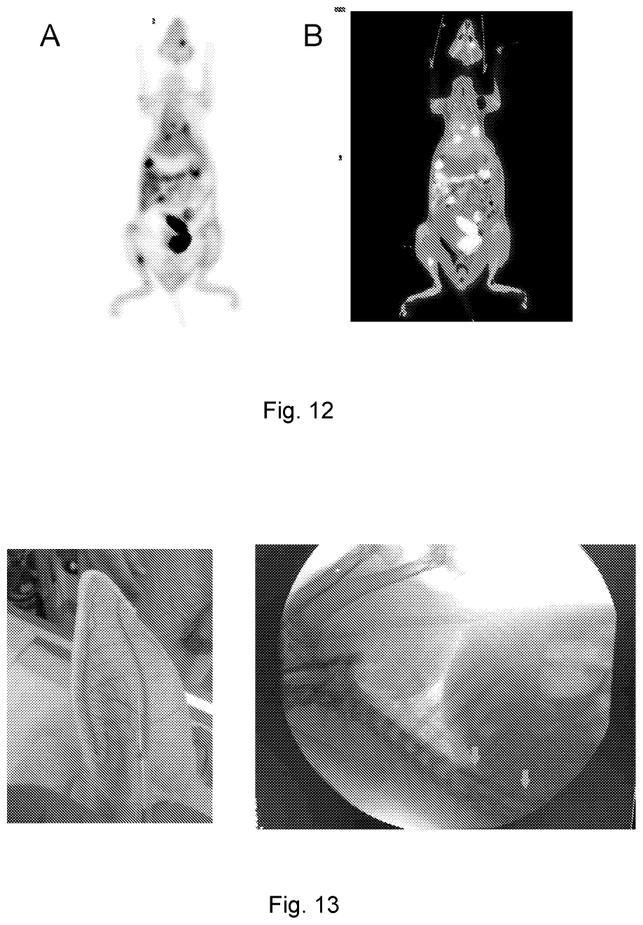

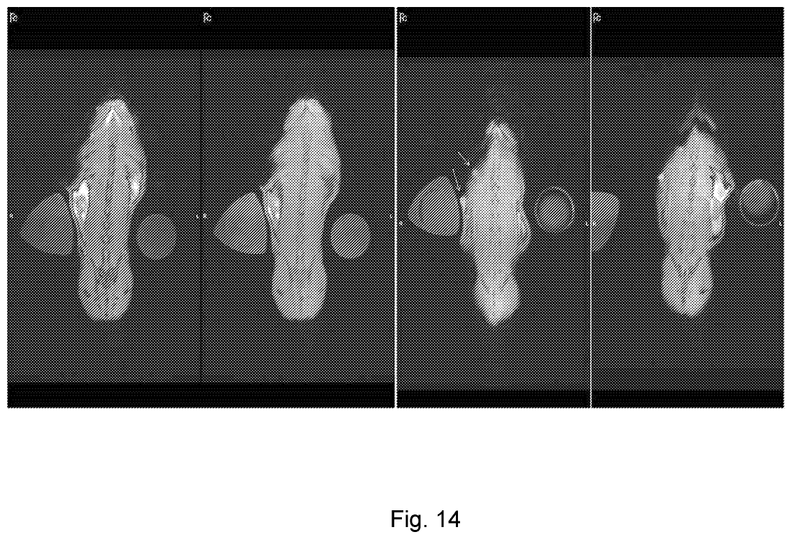

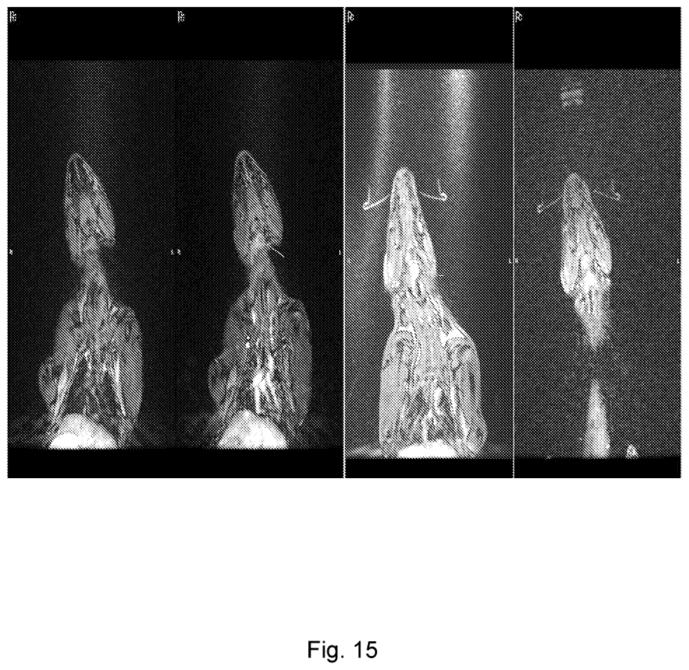

To test 3Gd(DOTA)-HPPH as a tumor imaging agent, rabbits were studied, each with hundreds of widespread, minute, metastatic anaplastic VX2 rabbit tumors (FIGS. 11, A, B and 12). VX2 was used because successful imaging of a rabbit tumor in a rabbit, as opposed to a xenograft, guarantees successful tumor imaging, not imaging of a host immune response. Each rabbit was first scanned by conventional F-18-FDG-PET/CT, and, immediately thereafter, injected with our tumor-avid MRI contrast medium, 3Gd(DOTA)-HPPH.

Original Metastatic Tumor model: to create widespread VX2 metastases, a tumor-cell suspension (8-15×10⁷ cells/mL 0.5 mL) retrograde was forcefully hand-injected into the central auricular artery (FIG. 13 A), and, at the same sitting, prior to the tumor cell injection, radiographic contrast medium (5 mL Iohexol) was injected into the central auricular artery under fluoroscopy, confirming visualization of the aorta (FIG. 13 B, arrows) and allowing the injecting physician to practice the injection force needed to achieve retrograde flow into the aorta.

Thus, the injected cells were disseminated systemically, by arterial blood flow, into multiple unpredictable locations. This method mirrors the real-world problem of defining widespread metastases in locations unknown, instead of defining tumors at sites where they have been placed by the investigator (an all-too-common practice in experimental animal tumor imaging.) When tumor implants were visible on CT, testing of 3Gd(DOTA)HPPH in comparison to F-18-FDG-PET/CT (the current gold standard) was ready.

VX2 implants are rabbit-to-rabbit growths, rather than xenografts-e.g., human Ward colon carcinoma cells implanted into animals; this factor guarantees that, when using this tumor model, tumor-avidity of any scanning agent (F-18-FDG, 3Gd(DOTA)-HPPH, or other compounds) is based upon the characteristics of the agent and the tumor only, rather than host vs. tumor immunologic factors seen in some xenografts.

Necropsy of dissected animals by a veterinary pathologist revealed that all visualized sites represent VX2 tumor deposits with very little necrosis; thus, the tumor-seeking agent had localized in living tumor.

Thus, unlike standard MRI and CT “tumor-avid” systemically-administered contrast media (small molecules that enter tumor through disrupted capillary beds and reside briefly in tumor interstitial space, and subsequently wash-out as blood levels of contrast medium fall via renal excretion), 3Gd (DOTA)-HPPH, based upon HPPH, a compound originally created for photodynamic therapy, accumulates slowly and progressively in tumors, “fading out” after 48 hours.

Therefore, in a human being, a lengthy total-body MRI, performed 24-h after I.V. injection of 3-Gd-(DOTA)HPPH, would not suffer from tumor “conspicuity fade” towards the end of a scan, and, tumors defined by this technique would remain visible long enough for subsequent biopsy under MRI guidance.

### Techniques

PET/CT imaging: 0.93 mCi (34.41 Mbeq) of Fluorine-18-fluoroxy-glucose (FDG) injected intravenously; animals were imaged at 30 min (GE Discovery ST, FOV 25 cm).

3Gd(DOTA)-HPPH dose: 10 μmol/kg (30 μmol of Gd/kg): molar dose of Gd/kg is one-tenth that which is used for a standard Gadolinium-enhanced clinical Mill study.

MRI technique: 1.5 Tesla (GE Sigma HD XT 1.5). Imaging at baseline, in the vascular phase, and at 24 and 48 h, 8 channel Body Coil. For the head and neck, coronal fat-sat T2 FOV 28 cm and coronal LAVA and post-contrast FOV 28 images were generated; these sequences were repeated for the body with a FOV of 48.

VX2 rabbit tumor cells injected retrograde in the auricular artery to achieve systemic dissemination: 8-15×10⁷ cells/mL, 0.5 mL.

Source of VX2 cells: The M. D. Andersen Cancer Center, Houston, Tex.

Rabbits: New Zealand White, male, weight 2.5 to 3.1 kg., albino.

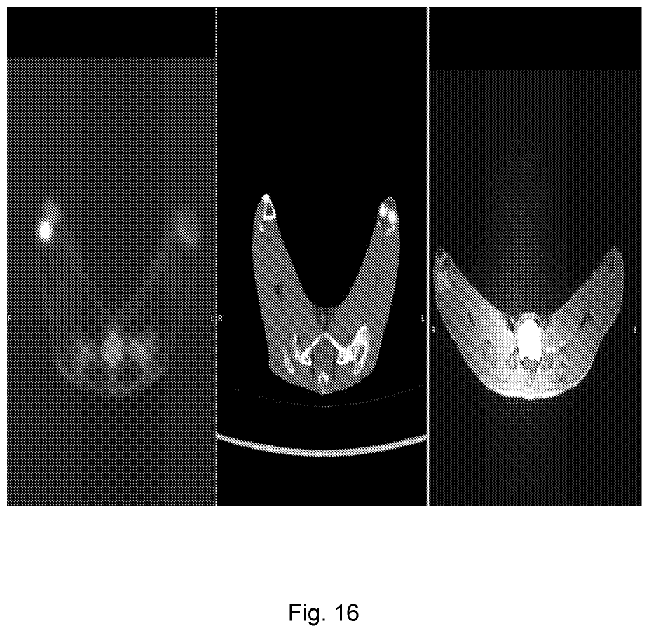

FIG. 16 shows virtually concurrent trans-axial images, through the rabbit pelvis and part of the leg, reveals the strengths and weaknesses of three high-imaging-technology systems.

To date, approximately 70 PET/MRI units have been installed worldwide, most of them Siemens, and a few Philips and GE. These units cost between $4,000,000 and $5,000,000, and annual maintenance generally adds another 10% to the cost; each unit requires 2-3 technologists. Moreover, each unit can scan only 10 patients per day, whereas a state-of-the-art FDG/PET/CT can generate four scans per hour.

Moreover, at least two interpreters are required for each total-body study: an MRI-trained radiologist and a nuclear medicine specialist; in fact, in major academic centers neuroradiogists now advocate for neuroradiology interpretation of the brain, head, and neck portions of the MRI/PET, so that the total number of readers may rise to THREE (which, of course, increases cost to both payers and healthcare providers.) Thus: reimbursement is a challenge. The situation is becoming ponderous and expensive.

The justification for MRI/PET units is that they provide exquisite resolution of soft tissue (MRI) and enhanced tumor conspicuity (FDG tumor-avidity). Notwithstanding this argument, 3(Gd)-DOTA-HPPH, can achieve both goals with a single MRI scanner and a single dose of contrast agent (and NO radiation exposure, not even the minimal exposure of PET).

The images 2-3 MRI tumors in rabbits were referred to as “MRI/PET WITHOUT THE PET.” The implications of this proposition are enormous: not simply obviation of the need for MRI/PET machines, but possibly even replacement of many PET/CT scans by total body MRI with a truly tumor-avid contrast medium, not to mention replacement of conventional MRI contrast media (e.g., Gadovist® and Magnevist®).

### Optical Imaging and MRI

Multimodality imaging refers to the combination of two or more imaging modalities with varying strengths in concurrently extracting physiological and anatomical information of living subjects in complimentary fashion (referred to as co-registration), so as to compensate for their individual weaknesses in pursuit of better accuracy and enrichment of information. Two particular combinations, namely PET with CT, and PET with MRI, have already seen clinical applications, while other combinations are currently being developed or used in pre-clinical settings. Eventually, maturation of multimodality imaging will revolutionize the way researchers interrogate complex biological systems, and will translate into diversified applications in the clinics.

Another combination of imaging modalities that can complement each other well is optical imaging and MRI. Optical imaging uses a non-invasive propagation of visible or near infra-red (IR) range light through tissue to develop an image and avoids the DNA damaging side effects associated with imaging modalities employ ionizing radiation, such as CT or PET. Other advantages of optical imaging include fast acquisition, high sensitivity, and lower cost. However, optical imaging has very poor resolution and limited depth of penetration. These shortfalls could be overcome when combining optical imaging with MRI.

On the other hand, the rationale for considering optical imaging within MRI roots can be stated from several perspectives. First of all, MRI is an expensive modality: the investment of the machinery, the cost of running a superconducting high-field magnet continuously, as well as the cost of the expertise required to run the clinical exams. Therefore, it makes great sense to incorporate additional devices to MRI facility that will provide more information to improve diagnosis or staging. Secondly, MRI cannot be used frequently on the subjects because of the cost, or because the subjects are not eligible (e.g. infants, patients with claustrophobia), in which case MRI can only be used sparingly. In these situations, optical imaging can supplement the MRI exams on a regular basis if daily or weekly monitoring is needed to track disease progression or response to therapy since optical imaging is less time-consuming and much cheaper.

This example described the development organ and tumor specific contrast media for magnetic resonance imaging (MRI) and Photodynamic Therapy (PDT). Currently, MRI is performed either without any intravenously-injected chemicals or after intravenous injection of Gadolinium-diethyline-triamine pentacetic-acid (Gd-DTPA). In lay terms, Gd-DTPA is a dye, which circulates briefly and is then excreted by the kidneys. During Gd-DTPA's circulation, MR images reveal increased brightness or signal from all tissues that are highly vascular, including lesions with a disrupted capillary like aggressive tumors, or areas of inflammation or dead tissue. These regions display enhanced signal compared to surrounding normal tissues. The enhanced brightness is due to the paramagnetic effect of Gadolinium on tissue water. We hope to modify the molecular structure of agents specifically developed for PDT, so that they can be bound to Gd to create tumor specific imaging agents for MRI. Then, after successful MRI, the tumors are treated with light, to demonstrate that the modified PDT agents remain photoactive and useful for PDT. The conjugation of tumor-specific photosensitizers (PS) with Gd chelates (Gd-DTPA) will have following advantages: (i) In contrast to Gd-DTPA, the corresponding PS-Gd (I-III) DTPA will retain in tumors for a longer period of time, which will help in monitoring the tumor response, (ii) a lower dose will be required for imaging,(iii) the same conjugate can be used for MRI and fluorescence imaging and both techniques are complimentary to each other, finally (iv) can also be used for image guided surgery with an option of PDT.

To date, there is no available MR contrast media that are organ-specific or tumor specific which concentrate in particular organs or tumors. The goal was to modify the molecular structure of agents, specifically developed for PDT so that they can be bound to Gd or radionuclides, to create tumor avid imaging agents for MRI or nuclear scanning.

The tumor uptake efficacy of HPPH-1Dota-1Gd, HPPH-2Dota-2Gd, and HPPH-3Dota-3Gd imaging agents compared in F344/Rats bearing ward rat colon tumor and in Balb/c mice bearing colon 26 tumors.

### Materials and Methods

### Materials

Rats: F344/NHsd (Female); Mice: Balb/c mice (Female)

### Tumor: Ward Rat Tumor: Colon 26

### Drugs: HPPH-1Dota-1Gd; HPPH-2Dota-2Gd; HPPH-3Dota-3Gd

### Methods:

Route of drug administration. The compounds (formulations) were injected intravenously (iv).

Drug Preparation and Schedules. HPPH-1Dota-1Gd HPPH-2Dota-2Gd and HPPH-3Dota-3Gd was synthesized and formulated. The formulations were given intravenously and the whole body images of tumored rats and mice were obtained at variable time points.

Doses. The formulations were used as a dose of 5 and 10 umol/kg concentration. The concentrations of the injected solutions were confirmed spectrophotometrically.

Tumor transplantation. A) Rats: The ward rat tumor cells were used for imaging. The cells were transplanted by injecting intraperitoneally. Treatment was initiated on between day 14-21 post transplantation. The cells were suspended at a density of 2-3×10⁶/ml in normal saline solution and see the growth of these cells for about 1-3 weeks.

The rats were injected with HPPH-DOTA-Gd (III) based MRI agents linked to target specific moieties imaging of the rats done under IVIS and MRI simultaneously to see the uptake of the imaging agents by tumors in the abdomen and lymph nodes. This will help to identify later the imaging guided therapy in metastatic models.

The rats were treated following an animal protocol approved by RPCI IACUC committee.

b). Mice: The cells were transplanted by injecting subcutaneously. Treatment was initiated 6-7 days later when tumor sizes (weight) reached approximately 3-5 mm. The Colon 26 cancer cells were suspended at a density of 1×10⁶/ml in normal saline solution. The tumors let to grow for 1 week until they achieve the desired size for treatment. The mice were treated following the animal protocol approved by RPCI IACUC committee.

Optical Imaging. Rats—After injecting the formulations, the tumored rats were imaged at 24 hours post-injection post necropsy. Mice—After injecting the formulations, the tumored mice were imaged at 1,2,3,4, 8, 12, 24, 48, 72 and 96 hours post-injection.

### Results

### Imaging. In Vivo Optical Imaging

Near-infrared optical imaging of tumor-bearing mice was performed at various time points following iv injection of the compound solution. The multi-spectral imaging system, IVIS Spectrum (Perkin-Elmer) along with Living Image (image acquisition and analysis software) is used. IVIS Spectrum has the capability to use either trans-illumination (from the bottom) or epi-illumination (from the top) to illuminate in vivo fluorescent sources. The instrument is equipped with 10 narrow band excitation filters (30 nm bandwidth) and 18 narrow band emission filters (20 nm bandwidth) that assist in significantly reducing auto fluorescence by the spectral scanning of filters and the use of spectral un mixing algorithms. Regions of interest (ROI) are defined for areas of compound accumulation (tumor, liver, skin,) and total and average signal within the region are recorded. Fluorescent intensity is expressed as the total radiant efficiency ([p/s]/[μW/cm²]. Results are expressed as mean total radiant efficiency.

Rats Imaging. The tumor-imaging of the formulation were looked at. The images were recorded at 24 hours post injection of the PS. The necropsy of the treated rats are done and abdomen showing tumor cell nodules, uptake of the HPPH-3Dota-3Gd. The data were plotted in comparing the accumulation of HPPH-3Dota-3Gd in tumors mimicking metastatic model. The peak concentrations in the HPPH alone are usually at 24 hours and the results correlated with our previous findings.

Mice Imaging. The tumor-imaging of the formulation were looked at. The images were recorded at 30 min, 1, 2, 3, 4, 5, 6, 7, 8, 24, 48, 72, and 96 hours post injection of the PS. The data were plotted in comparing the accumulation of PS in tumor, liver and skin.

The tumor accumulation data obtained from HPPH-3DOTA-3Gd formulation definitely show more accumulation in rats after necropsy bearing ward rat tumor cells in the peritoneal micro metastasis at 24h post-injection. The data was duplicated in 3 different rats and showed the uptake/accumulations in the same pattern. The results were also simultaneously compared with MRI on same rats which showed similar enhancement of tumors with imaging agents and also at earlier time points.

In vivo fluorescence data indicate highly significant uptake of HPPH-3DOTA-3Gd in this rat model mimicking metastatic model. Thus these findings warrant potential of these agents as MRI contrast agents in clinical settings.

The fluorescence uptake of HPPH-DOTA-Gd (I-III) based MRI agents was done in mice bearing colon 26 tumors transplanted subcutaneously. The HPPH-DOTA-Gd I and II peaks between 1 and 24 hour (Ex:640 nm/Em:680 nm).The HPPH-DOTA-Gd III showed enhanced and significant uptake and stays in tumors longer (Ex:640 nm/Em:680 nm) and clears 80-90% from liver by 24 hours. Thus, a direct correlation between the tumor-uptake of the different imaging agents was observed in this tumor model.

### In Vitro PDT Efficacy

The in vitro photosensitizing efficacy of HPPH conjugated with mono-, di- and tri-Gd(DOTA) chelate was determined by MTT assay. In brief, Colon26 cells were plated into 96 well plates (3600 cells per well), allowed to adhere to the plate and are treated with a range of compound concentrations for 24 hours. After the 24 hours drug incubation, cells were irradiated with light at 665 nm. After 48 hours post exposure of light, MTT [3-(4,5-dimethylthiazol)-2-yl)-2,5-diphenyl tetrazolium bromide] was added to each well. The solubilized formazan product (by the addition of DMSO) was measured spectrophotometrically using a BIOTEK® plate reader. Data is processed using GENS software. A medium blank value is subtracted from all samples and optical density (O. D.) values of treated cells are divided by mean O. D. value of untreated cells for each light dose. Results are expressed as the percent cell survival +/−SD and are plotted as % control vs. concentration of compound or % of control vs light dose using Graph Pad Prism %. The results summarized in FIG. 20 clearly show that all conjugates are significantly effective PDT agents.

Histopathology Report. Received by IDEXX Reference Laboratories, Inc. were formalin-fixed samples of heart, lung, kidney, spleen, liver and skin from twelve rats on Roswell Park Study ID Pandey-14-2017, IDEXX Reference Laboratories, Inc. Study Accession 70556-2017. The animals were divided into two treatment groups (Day 8 Low Dose, males and females; Day 28 Low Dose, males and females) with three males and three females per group. Submitted tissues were trimmed, processed, blocked, sectioned, stained with H&E and examined microscopically. Observed microscopic changes were graded, as to severity, utilizing a standard grading system whereby 0=no significant change, 1=minimal, 2=mild, 3=moderate and 4=severe.

Microscopic findings are summarized by group and sex in Table 5: Summary of Histopathological Findings. Changes for each animal are given in Tables 6-13: Individual Animal Histopathology Report. Probable, treatment-related changes were noted in the spleen of both males and females at Day 8 and were characterized by elevated levels of extramedullary hematopoiesis which returned to normal levels at Day 28. Remaining observed changes were either terminal events associated with animal sacrifice (i.e. minimal to mild hemorrhage in the lungs) or common findings in the rat (i.e. minimal to mild mineralization in the kidneys of females and extramedullary hematopoiesis in the liver)

TABLE 5
Summary of Histopathological Findings.

| | Males | Males | Females | Females |
|---|---|---|---|---|
| | Day 8 | 28-Day | Day 8 | 28-Day |
| Group | Low Dose | Low Dose | Low Dose | Low Dose |
| Number of Animals | 3 | 3 | 3 | 3 |
| Heart: | | | | |
| No significant changes | 3 | 3 | 3 | 3 |
| Lungs | | | | |
| No significant changes | 1 | 0 | 2 | 3 |
| Hemorrhage | 2 (2.0) | 3 (1.3) | 1 (1.0) | 0 |
| Kidneys | | | | |
| No significant changes | 3 | 2 | 2 | 2 |
| Mineralization | 0 | 0 | 1 (1.0) | 1 (2.0) |
| Protein casts | 0 | 1 (1.0) | 0 | 0 |
| Spleen | | | | |
| Extramedullary hematopoiesis | 3 (3.0) | 3 (1.3) | 3 (1.7) | 3 (1.0) |
| Liver | | | | |
| No significant changes | 2 | 1 | 2 | 2 |
| Extramedullary hematopoiesis | 1 (1.0) | 2 (1.0) | 1 (1.0) | 1 (2.0) |
| Skin: No significant changes | 3 | 3 | 3 | 3 |

TABLE 6
Individual Animal Histopathology Reports
Group-Day 8, Imaging Dose (Male)

| | |
|---|---|
| Animal Number-1: | |
| Heart: | No significant change noted. |
| Lungs: | No significant change noted. |
| Kidneys: | No significant change noted. |
| Spleen: | Extramedullary hematopoiesis, moderate. |
| Liver: | No significant change noted. |
| Skin: | No significant change noted. |
| Animal Number-2: | |
| Heart: | No significant change noted. |
| Lungs: | Hemorrhage, multifocal, mild. |
| Kidneys: | No significant change noted. |
| Spleen: | Extramedullary hematopoiesis, moderate. |
| Liver: | Extramedullary hematopoiesis, multifocal, minimal. |
| Skin: | No significant change noted. |

TABLE 7
Individual Animal Histopathology Reports
Group-Day 8, Imaging Dose (Male)

| | |
|---|---|
| Animal Number-3: | |
| Heart: | No significant change noted. |
| Lungs: | Hemorrhage, multifocal, mild. |
| Kidneys: | No significant change noted. |
| Spleen: | Extramedullary hematopoiesis, moderate. |
| Liver: | No significant change noted. |
| Skin: | No significant change noted. |

TABLE 8
Individual Animal Histopathology Reports
Group-Day 28, Imaging Dose (Male)

| | |
|---|---|
| Animal Number-10: | |
| Heart: | No significant change noted. |
| Lungs: | Hemorrhage, multifocal, minimal. |
| Kidneys: | No significant change noted. |
| Spleen: | Extramedullary hematopoiesis, minimal. |
| Liver: | Extramedullary hematopoiesis, |
| | multifocal, minimal. |
| Skin: | No significant change noted. |
| Animal Number-11: | |
| Heart: | No significant change noted. |
| Lungs: | Hemorrhage, focal, minimal. |
| Kidneys: | No significant change noted. |
| Spleen: | Extramedullary hematopoiesis, mild. |
| Liver: | Extramedullary hematopoiesis, minimal. |
| Skin: | No significant change noted. |

TABLE 9
Individual Animal Histopathology Reports
Group-Day 28, Imaging Dose (Male)

| | |
|---|---|
| Animal Number-12: | |
| Heart: | No significant change noted. |
| Lungs: | Hemorrhage, multifocal, mild. |
| Kidneys: | Protein casts, focal, minimal. |
| Spleen: | Extramedullary hematopoiesis, minimal. |
| Liver: | No significant change noted. |
| Skin: | No significant change noted. |

TABLE 10
Individual Animal Histopathology Reports
Group-Day 8, Imaging Dose (Female)

| | |
|---|---|
| Animal Number-16: | |
| Heart: | No significant change noted. |
| Lungs: | Hemorrhage, multifocal, mild. |
| Kidneys: | No significant change noted. |
| Spleen: | Extramedullary hematopoiesis, mild. |
| Liver: | No significant change noted. |
| Skin: | No significant change noted. |
| Animal Number 17: | |
| Heart: | No significant change noted. |
| Lungs: | No significant change noted. |
| Kidneys: | Mineralization, multifocal, minimal. |
| Spleen: | Extramedullary hematopoiesis, minimal. |
| Liver: | No significant change noted. |
| Skin: | No significant change noted. |

TABLE 11
Individual Animal Histopathology Reports
Group-Day 28, Imaging Dose Female

| | |
|---|---|
| Animal Number-18: | |
| Heart: | No significant change noted. |
| Lungs: | No significant change noted. |
| Kidneys: | No significant change noted. |
| Spleen: | Extramedullary hematopoiesis, mild. |
| Liver: | Extramedullary hematopoiesis, focal, minimal. |
| Skin: | No significant change noted. |

TABLE 12
Individual Animal Histopathology Reports
Group-Day 28, Imaging Dose (Female)

| | |
|---|---|
| Animal Number-25: | |
| Heart: | No significant change noted. |
| Lungs: | No significant change noted. |
| Kidneys: | No significant change noted. |
| Spleen: | Extramedullary hematopoiesis, minimal. |
| Liver: | Extramedullary hematopoiesis, multifocal, mild. |
| Skin: | No significant change noted. |
| Animal Number-26: | |
| Heart: | No significant change noted. |
| Lungs: | No significant change noted. |
| Kidneys: | No significant change noted. |
| Spleen: | Extramedullary hematopoiesis, minimal. |
| Liver: | No significant change noted. |
| Skin: | No significant change noted. |

TABLE 13
Individual Animal Histopathology Reports
Group-Day 28, Imaging Dose (Female)

| | |
|---|---|
| Animal Number-27: | |
| Heart: | No significant change noted. |
| Lungs: | No significant change noted. |
| Kidneys: | Mineralization, multifocal, mild. |
| Spleen: | Extramedullary hematopoiesis, minimal. |
| Liver: | No significant change noted. |
| Skin: | No significant change noted. |

Although the present disclosure has been described with respect to one or more particular embodiments and/or examples, it will be understood that other embodiments and/or examples of the present disclosure may be made without departing from the scope of the present disclosure.

1. A compound comprising:
F-L-R,
wherein F is a tetrapyrrole group, optionally, comprising a chelating ion, L is a linker, and R is a ligand, optionally, comprising a Gd(III) ion coordinated to the ligand.

2. The compound of claim 1, wherein F is chosen from: 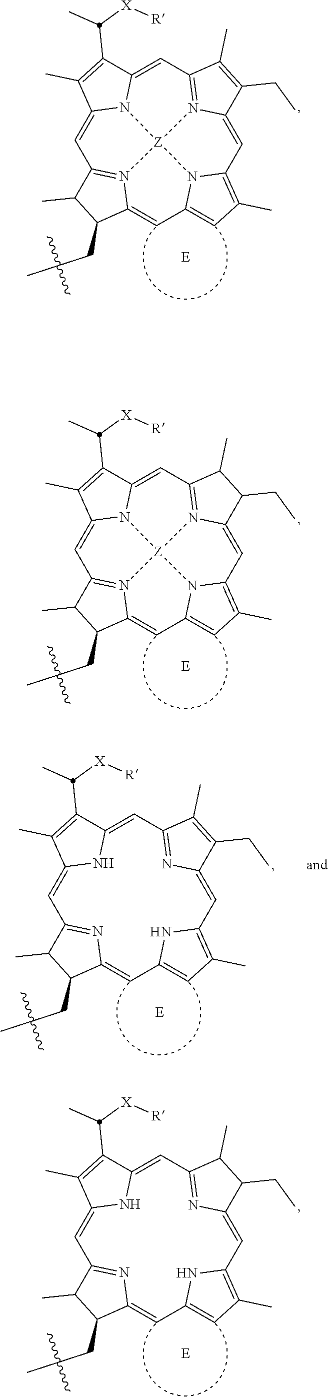
wherein R′ is an alkyl group or 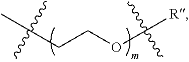
wherein m is an integer from 1 to 12, or heteroalkyl group;
X is chosen from O, S, and NH;
Z is chosen from ²H, ⁶⁴Cu, ¹¹¹In, and ⁵⁷Ga;
E is chosen from (i) a five-member isocyclic ring having a ketone and (ii) a six member N-substituted ring, wherein the N is functionalized with a substituent chosen from an alkyl group, a heteroalkyl group, a peptide group, and 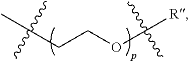
wherein p is an integer from 1 to 12;
R″ is at each occurrence independently chosen from an alkyl group, a peptide group, and a heteroalkyl group; and
the dotted carbon is either chiral or achiral.

3. The compound of claim 2, wherein E is: 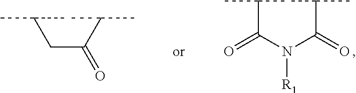
wherein R₁ is chosen from an alkyl group, a heteroalkyl group, a peptide group, and 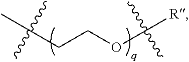
wherein q is an integer from 1 to 12 and R″ is chosen from an alkyl group, a peptide group, and a heteroalkyl group.

4. The compound of claim 1, wherein L is chosen from:

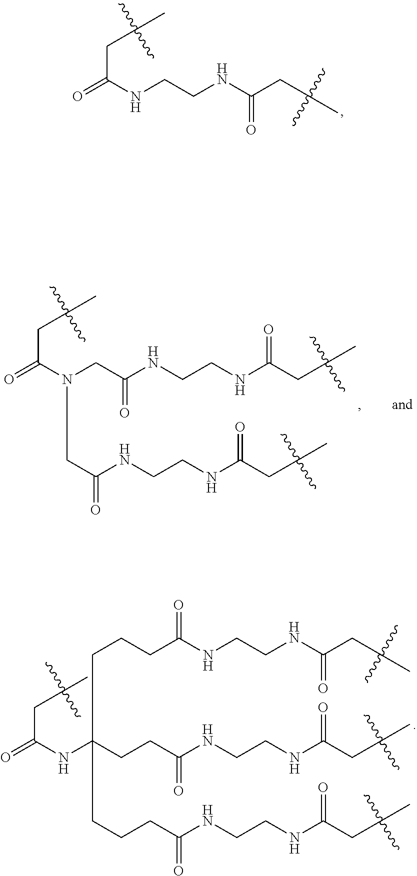 [SMILES: *CC(=O)NCCNC(=O)C* ; *CC(=O)NCCNC(=O)CN(CC(=O)NCCNC(=O)C*)C(=O)C* ; *CC(=O)NCCNC(=O)CCCC(CCCC(=O)NCCNC(=O)C*)(CCC(=O)NCCNC(=O)C*)NC(=O)C*]

5. The compound of claim 1, wherein R is chosen from: 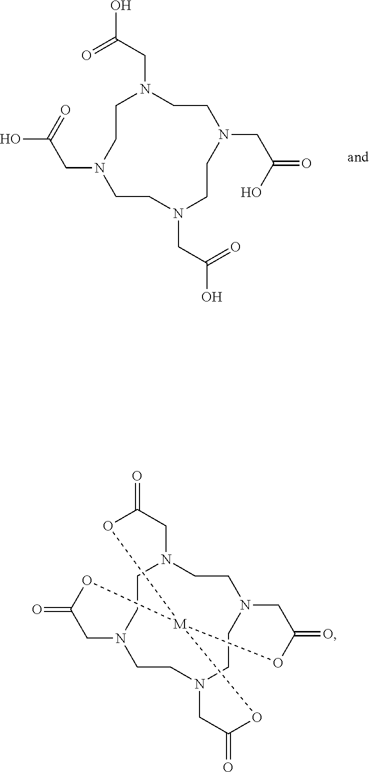
wherein M is a Gd(III) ion.

6. The compound of claim 1, wherein the compound is: 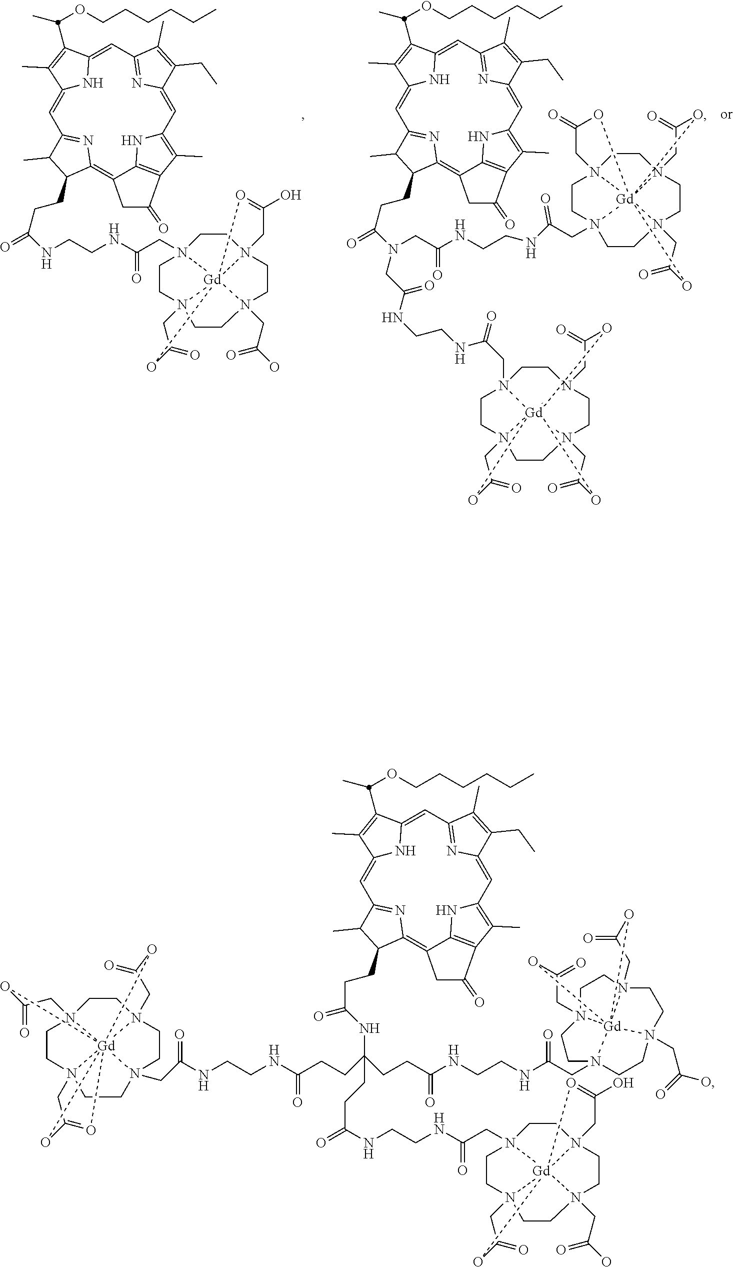
wherein the dotted carbon is either chiral or achiral.

7. A composition comprising one or more compound claim 1.

8. The composition of claim 7, wherein the composition further comprises a pharmaceutically acceptable carrier.

9. A method for detecting the presence of a hyperproliferative tissue in an individual comprising:
administering to the individual an effective quantity of one or more compound of claim 1 and/or one or more composition, each individual composition comprising one or more compound of claim 1; and
imaging the individual or a portion thereof to detect the presence or absence of a hyperproliferative tissue in an individual.

10. The method of claim 9, wherein the imaging is magnetic resonance (MR) imaging and/or fluorescence imaging.

11. The method of claim 9, wherein the method further comprises:
exposing the individual with light of a wavelength to kill or impair the hyperproliferative tissue.

12. The method of claim 11, wherein the compound(s) selectively interact(s) with hyperproliferative tissue relative to normal tissue, and exposing the subject with light of a wavelength to kill or impair the hyperproliferative tissue.

13. The method of claim 12, further comprising allowing time for any of the compound(s) that is/are not selectively interacted with the hyperproliferative tissue to clear from the normal tissue of the subject prior to the step of exposing.

14. The method of claim 9, wherein the hyperproliferative tissue is a vascular endothelial tissue, a neovasculature tissue, a neovasculature tissue present in the eye, an abnormal vascular wall of a tumor, a solid tumor, a tumor of a head, a tumor of a neck, a tumor of an eye, a tumor of a gastrointestinal tract, a tumor of a liver, a tumor of a breast, a tumor of a prostate, a tumor of a lung, a nonsolid tumor, and malignant cells of one of a hematopoietic tissue and a lymphoid tissue.

15. The method of claim 9, wherein the administering comprises reconstituting the one or more compound and/or the one or more composition; and administering the reconstituted compound(s) and/or the composition(s) to the individual.

16. The method of claim 15, wherein the one or more compound is one or more lyophilized compound and/or one or more composition comprises one or more lyophilized compound.

17. A method of photodynamic therapy for treating hyperproliferative tissue in an individual, comprising:
administering to the individual a compound of claim 1 that selectively interacts with the hyperproliferative tissue relative to normal tissue, and
exposing the individual with light of a wavelength to activate the compound, whereby the hyperproliferative tissue is treated.

18. The method of claim 17, further comprising imaging the individual or a portion thereof to detect the presence or absence of a hyperproliferative tissue in the individual.

19. The method of claim 18, wherein the imaging is MR imaging and/or fluorescence imaging.

20. The method of claim 19, wherein the hyperproliferative tissue is a vascular endothelial tissue, a neovasculature tissue, a neovasculature tissue present in the eye, an abnormal vascular wall of a tumor, a solid tumor, a tumor of a head, a tumor of a neck, a tumor of an eye, a tumor of a gastrointestinal tract, a tumor of a liver, a tumor of a breast, a tumor of a prostate, a tumor of a lung, a nonsolid tumor, and malignant cells of one of a hematopoietic tissue and a lymphoid tissue.

21. The method of claim 17, wherein the administering comprises reconstituting the compound; and administering the reconstituted compound to the individual.

22. The method of claim 21, wherein the one or more compound is one or more lyophilized compound and/or one or more composition comprises one or more lyophilized compound.

23. A kit comprising
one of more compound of claim 1 and/or one or more composition, each individual composition comprising one or more compound of claim 1; and
instructions for use of the one or more compound and/or the one or more composition.
